SEQUENCE LISTING (15 sequences)

SEQ ID NO: 1 - protein, 9 aa, Artificial Sequence
  compound 1
  misc_feature (2)..(2): Xaa=D-Lys
  misc_feature (4)..(4): Xaa=NMe-Phe
YXGXDPQRF

SEQ ID NO: 2 - protein, 9 aa, Artificial Sequence
  compound 2
  misc_feature (2)..(2): Xaa=D-Lys
  misc_feature (4)..(4): Xaa=NMe-Phe
YXGXEPQRF

SEQ ID NO: 3 - protein, 9 aa, Artificial Sequence
  compound 3
  misc_feature (4)..(4): Xaa=NMe-Phe
YKGXDPQRF

SEQ ID NO: 4 - protein, 9 aa, Artificial Sequence
  compound 4
  misc_feature (4)..(4): Xaa=NMe-Phe
  misc_feature (5)..(5): Xaa=D-Asp
YKGXXPQRF

SEQ ID NO: 5 - protein, 9 aa, Artificial Sequence
  compound 5
  misc_feature (2)..(2): Xaa=D-Lys
  misc_feature (4)..(4): Xaa=NMe-Phe
  misc_feature (5)..(5): Xaa=D-Asp
YXGXXPQRF

SEQ ID NO: 6 - protein, 9 aa, Artificial Sequence
  compound 6
  misc_feature (2)..(2): Xaa=D-Asp
  misc_feature (4)..(4): Xaa=NMe-Phe
YXGXKPQRF

SEQ ID NO: 7 - protein, 9 aa, Artificial Sequence
  compound 7
  misc_feature (2)..(2): Xaa=D-Glu
  misc_feature (4)..(4): Xaa=NMe-Phe
YXGXKPQRF

SEQ ID NO: 8 - protein, 9 aa, Artificial Sequence
  compound 8
  misc_feature (2)..(2): Xaa=D-Orn
  misc_feature (4)..(4): Xaa=NMe-Phe
YXGXDPQRF

SEQ ID NO: 9 - protein, 9 aa, Artificial Sequence
  compound 9
  misc_feature (2)..(2): Xaa=D-Dab
  misc_feature (4)..(4): Xaa=NMe-Phe
YXGXDPQRF

SEQ ID NO: 10 - protein, 9 aa, Artificial Sequence
  compound 10
  misc_feature (2)..(2): Xaa=D-Dap
  misc_feature (4)..(4): Xaa=NMe-Phe
YXGXDPQRF

SEQ ID NO: 11 - protein, 9 aa, Artificial Sequence
  compound 11
  misc_feature (2)..(2): Xaa=D-Orn
  misc_feature (4)..(4): Xaa=NMe-Phe
YXGXEPQRF

SEQ ID NO: 12 - protein, 9 aa, Artificial Sequence
  compound 12
  misc_feature (2)..(2): Xaa=D-Lys
  misc_feature (4)..(4): Xaa=NMe-Phe
YXGXDGQRF

SEQ ID NO: 13 - protein, 9 aa, Artificial Sequence
  compound 13
  misc_feature (2)..(2): Xaa=D-Lys
  misc_feature (4)..(4): Xaa=NMe-Phe
  misc_feature (7)..(7): Xaa=beta-Ala
  misc_feature (9)..(9): Xaa=Cha
YXGXDPXRX

SEQ ID NO: 14 - protein, 9 aa, Artificial Sequence
  compound 14
  misc_feature (2)..(2): Xaa=D-Lys
  misc_feature (4)..(4): Xaa=NMe-Phe
  misc_feature (7)..(7): Xaa=Aib
  misc_feature (9)..(9): Xaa=Cha
YXGXDPXRX

SEQ ID NO: 15 - protein, 9 aa, Artificial Sequence
  DN-9
  misc_feature (2)..(2): Xaa=D-Ala
  misc_feature (4)..(4): Xaa=NMe-Phe
YXGXGPQRF

(19) United States
(12) Patent Application Publication
(10) Pub. No.: US 2024/0166690 A1
(43) Pub. Date: May 23, 2024

(54) MULTI-TARGET CYCLOPEPTIDE MOLECULE FOR OPIOID/NEUROPEPTIDE FF RECEPTORS, AND PREPARATION THEREFOR AND APPLICATION THEREOF

(71) Applicant: SHANGHAI TIANCI LIFE SCIENCE DEVELOPMENT CO., LTD., Shanghai (CN)

(72) Inventors: Quan FANG, Shanghai (CN); Mengna ZHANG, Shanghai (CN); Biao XU, Shanghai (CN); Run ZHANG, Shanghai (CN); Ning LI, Shanghai (CN)

(21) Appl. No.: 18/550,207

(22) PCT Filed: Feb. 22, 2022
(86) PCT No.: PCT/CN2022/077309

§ 371 Date: Sep. 12, 2023

(30) Foreign Application Priority Data
Mar. 12, 2021 (CN) 202110272260.4

(51) Int. Cl. C07K 7/64; A61K 38/00; A61P 25/04

(52) U.S. Cl. CPC C07K 7/64; A61P 25/04; A61K 38/00

(57) ABSTRACT

Provided is a novel peripherally restricted multi-target cyclopeptide molecule for an opioid receptor and a neuropeptide FF (NPFF) receptor, or a pharmaceutically acceptable salt thereof. By using DN-9 as a chemical template, structural optimization is performed on opioid peptide and NPFF pharrnacophores by means of polypeptide chemical strategies such as amino acid replacement and cyclization modification to obtain a series of cyclopeptide molecules. The cyclopeptide molecules can activate both opioid receptors and NPFF receptors, and the analgesic activity and the analgesic duration thereof are greatly increased compared with parent DN-9 molecules, and opioid side effects such as analgesic tolerance, constipation, and addiction are reduced.

### FIELD OF THE INVENTION

The present invention relates to the field of biochemical technology. Specifically, the present invention relates to a class of multi-target cyclopeptide molecules for opioidreceptors and neuropeptide FF receptors, and preparation therefor and application thereof

### BACKGROUND OF THE INVENTION

Morphine and fentanyl are the most common opioid analgesic drugs for the treatment of acute and chronic pain, but the side effects generated during long-term use severely limit their clinical application, such as respiratory suppression, tolerance, abuse, and constipation. Therefore, it is of great significance to explore and develop new opioid analgesic drugs with high efficiency and low side effects.

In recent years, new opioid analgesic drugs developed using strategies such as multi-target and peripherally restricted molecules can effectively reduce the side effects associated with traditional opioid drugs, and have potential application prospects in the development of efficient and low side effect analgesic drugs. For example, the multi-target agonists BU08028, AT-121, and BU10038 developed for targeting opioid/nociceptin (NOP) receptors have been shown to have effective analgesic effect in non-human primates with low adverse reactions; Cebranopadol, a multi-target agonist for opioid/NOR receptors, is in the phase II clinical study stage and is used for the treatment of pathological pain such aspostoperative pain, diabetes induced peripheral neuropathy, cancer pain. Its tolerance, addiction, respiratory inhibition and other side effects are significantly reduced than those of common opioids. Peripherally restricted Kappa-opioid receptor agonists Acemadolin and CR845 are currently in clinical research and used for the treatment of moderate to severe chronic kidney disease related pruritus and acute postoperative pain by intravenous administration, due to the inability of these molecules to activate central opioid receptors through the blood-brain barrier, their side effects are significantly reduced.

Neuropeptide FF (NPFF) is an opioid regulatory peptide that exhibits anti-opioid activity above the spinal cord level, and can produce opioid-like analgesic effects at the spinal cord level and enhance analgesic effects induced by opioids. In addition, NPFF is also involved in the regulation of side effects such as analgesic tolerance and addiction of opioid drugs (Brain Res. 1999, 848:191-196). Patent 201610252648.7 discloses a novel chimeric peptide DN-9 based on opioid peptides and NPFF. In vitro functional experiments have shown that the chimeric peptide acts as a multi-target agonist for opioid receptor and NPFF receptor. In vivo pharmacological data have shown that DN-9 produces stronger analgesic effects than that of morphine at both central level and peripheral level. In addition, compared to morphine, DN-9 significantly reduced opioid-like side effects such as addiction, analgesic tolerance, and constipation (J Med Chem. 2016, 59:10198-10208; J Pain. 2020, 21:477-493; Br J Pharmacol. 2020, 177:93-109). However, there is still a space for further improvement in the reported pharmacological properties of DN-9, such as its analgesic activity and analgesic duration.

Peptide drugs are a class of drugs with rapid market growth. As of now, nearly 100 peptide drugs have been approved for marketing globally, and about 200 new peptide drugs have entered the preclinical and clinical research stages. However, the instability of peptide drugs themselves is a key technical challenge that limits their further development. Previous studies have shown that chemical modification strategies such as non-natural amino acid substitution and modification, cyclization, polyethylene glycol modification, and glycosylation can effectively improve the stability, receptor selectivity, and bioavailability of peptide molecules, and further enhance their drug resistance.

Therefore, it is necessary to develop a peptide based analgesic drug with high efficiency, low side effects, and long-term analgesic effects.

### SUMMARY OF THE INVENTION

The main purpose of the present invention is to provide a multi-target cyclopeptide molecule for opioid receptors and neuropeptide FF receptors, or a pharmaceutically acceptable salt thereof.

Another purpose of the present invention is to provide a preparation method for the above multi-target cyclopeptide molecule or a pharmaceutically acceptable salt thereof.

Further purpose of the present invention is to provide therapeutic use of the above multi-target cyclopeptide molecule or a pharmaceutically acceptable salt thereof.

The first aspect of the present invention provides a multi-target cyclopeptide molecule for opioid receptors and neuropeptide FF receptors, or a pharmaceutically acceptable salt thereof, and the structure of the cyclopeptide molecule is shown in formula I:

Tyr-c^{[2,5]}[Xaa2-Gly-NMe-Phe-Xaa5]-Xaa6-Xaa7-Arg-Xaa9-NH₂ (I)
wherein,
Xaa2 is Lys, D-Lys, D-Asp, D-Glu, D-Orn, D-Dab, or D-Dap;
Xaa5 is Asp, D-Asp, Glu, or Lys;
Xaa6 is Pro or Gly;
Xaa7 is Gln β-Ala or Aib;
Xaa9 is Phe or Cha;
c^{[2,5]} represents the presence of a cycle-forming covalent bond between the two amino acid residues Xaa2 and Xaa5 in the amino acid sequence.

In another preferred embodiment, the cycle-forming covalent bond between the two amino acid residues Xaa2 and Xaa5 includes the formation of amide bond through dehydration condensation.

In another preferred embodiment, the structure of the cyclopeptide molecule is shown in formula II:

```
(II)
Tyr-Xaa2~L0~Xaa5-Xaa6-Xaa7-Arg-Xaa9-NH₂
        | |
        Gly-NMePhe
```

wherein,
Xaa2, Xaa5, Xaa6, Xaa7, and Xaa9 as defined above;
‘˜L0˜’ represents the cycle-forming covalent bond between the two amino acid residues Xaa2 and Xaa5.

In another preferred embodiment, the cyclopeptide molecule is selected from the following compound:

compound 1:
Tyr-c^{[2,5]}[D-Lys-Gly-NMe-Phe-Asp]-Pro-
Gln-Arg-Phe-NH₂
compound 2:
Tyr-c^{[2,5]}[D-Lys-Gly-NMe-Phe-Glu]-Pro-
Gln-Arg-Phe-NH₂
compound 3:
Tyr-c^{[2,5]}[Lys-Gly-NMe-Phe-Asp]-Pro-
Gln-Arg-Phe-NH₂
compound 4:
Tyr-c^{[2,5]}[Lys-Gly-NMe-Phe-D-Asp]-Pro-
Gln-Arg-Phe-NH₂
compound 5:
Tyr-c^{[2,5]}[D-Lys-Gly-NMe-Phe-D-Asp]-
Pro-Gln-Arg-Phe-NH₂
compound 6:
Tyr-c^{[2,5]}[D-Asp-Gly-NMe-Phe-Lys]-Pro-
Gln-Arg-Phe-NH₂
compound 7:
Tyr-c^{[2,5]}[D-Glu-Gly-NMe-Phe-Lys]-Pro-
Gln-Arg-Phe-NH₂
compound 8:
Tyr-c^{[2,5]}[D-Orn-Gly-NMe-Phe-Asp]-Pro-
Gln-Arg-Phe-NH₂
compound 9:
Tyr-c^{[2,5]}[D-Dab-Gly-NMe-Phe-Asp]-Pro-
Gln-Arg-Phe-NH₂
compound 10:
Tyr-c^{[2,5]}[D-Dap-Gly-NMe-Phe-Asp]-Pro-
Gln-Arg-Phe-NH₂
compound 11:
Tyr-c^{[2,5]}
[D-Orn-Gly-NMe-Phe-Glu]-Pro-Gln-Arg-
Phe-NH₂
compound 12:
Tyr-c^{[2,5]}
[D-Lys-Gly-NMe-Phe-Asp]-Gly-Gln-Arg-
Phe-NH₂
compound 13:
Tyr-c^{[2,5]}[D-Lys-Gly-NMe-Phe-Asp]-Pro-
β-Ala-Arg-Cha-NH₂;
and
compound 14:
Tyr-c^{[2,5]}[D-Lys-Gly-NMe-Phe-Asp]-Pro-
Aib-Arg-Cha-NH₂.

In another preferred embodiment, the cyclopeptide molecule as shown in formula I has one or more features selected from the group consisting of:
(1) the cyclopeptide molecule as shown in formula I is a dual target agonist for opioid receptors and NPFF receptors;
(2) the analgesic activity of subcutaneous injection of cyclopeptide molecule as shown in formula I is more than 10 times that of DN-9, preferably more than 100 times, more preferably more than 500 times, 1000 times, 10000 times, or 10⁶ times;
(3) the oral analgesic activity of the cyclopeptide molecule as shown in formula I is more than 3 times that of DN-9, preferably more than 10 times, more preferably more than 100 times, 500 times, 1000 times, or 10000 times;
(4) the cyclopeptide molecule as shown in formula I does not pass through the blood-brain barrier;
(5) the cyclopeptide molecule as shown in formula I has not shown analgesic tolerance after continuous oral administration for more than 5 days (preferably more than 8 days);
(6) the cyclopeptide molecule as shown in formula I does not have constipation side effect;
(7) the cyclopeptide molecule as shown in formula I does not have addictive side effect.

The second aspect of the present invention provides a preparation method for multi-target cyclopeptide molecule or a pharmaceutically acceptable salt thereof, comprising the steps of:
(a) using liquid phase synthesis method and/or solid phase synthesis method, synthesising the peptide chain according to the amino acid sequence corresponding to the structural formula I, thereby obtaining linear peptide chain; and
(b) cyclizing the linear peptide chain to obtain the multi-target cyclopeptide molecule for opioid receptors and neuropeptide FF receptors, or a pharmaceutically acceptable salt thereof.

In another preferred embodiment, the method further includes: separating the multi-target cyclopeptide molecule or a pharmaceutically acceptable salt thereof obtained in step (b) to obtain purified multi-target cyclopeptide molecule or a pharmaceutically acceptable salt thereof.

In another preferred embodiment, in step (b), the side chains of Xaa2 and Xaa5 are cyclized to form a cycle-forming covalent bond between the two amino acid residues Xaa2 and Xaa5.

In another preferred embodiment, in step (a), peptide chain synthesis is carried out from the C-terminus of the amino acid sequence to the N-terminus.

In another preferred embodiment, in step (a), peptide chain synthesis is carried out from the N-terminus of the amino acid sequence to the C-terminus.

In another preferred embodiment, in step (a), the solid phase synthesis method comprises the process steps selected from the group consisting of: pretreatment of the solid phase carrier, amino acid condensation, extension of the peptide chain, compression, drying, and cutting of the peptide, extraction, purification, and the combinations thereof.

In another preferred embodiment, the solid phase carrier is amino resin.

In another preferred embodiment, condensation reagents used in the amino acid condensation step include a combination of HOBt, HBTU, and DIEA.

In another preferred embodiment, cyclization reagent used in the cyclization step includes a combination of PyBOP and DIEA.

In another preferred embodiment, the preparation method comprises the process steps of:
i. Resin pretreatment: swelling a certain amount of Rink-Amide-MBHA resin in dichloromethane (DCM) for 30 minutes, extracting to dryness, and rinsing the resin with N, N-dimethylformamide (DMF). The volume mass ratio of DCM to resin is 8-12 mL/g. The mixture is tested by ninhydrin, the solution is light yellow and the resin is colorless under normal circumstances.
ii. performing amino acid condensation in turn;
ii.i Removing fluorene methoxycarbonyl (Fmoc) protecting groups: adding a mixture of 1,8-diazabicycloundecano-7-ene (DBU), piperidine, and DMF with a volume ratio of 1:1:98 into the resin obtained in step i. Stirring the reaction three times at 60-100 rpm, with the time of first two stirrings is 2-6 minutes and the time of last stirring is 8-12 minutes; preferably the time is 5 minutes for the first two stirrings and 10 minutes for the last one. Finally, washing with DMF. The mixture is tested by ninhydrin, the solution is blue purple and the resin is blue purple under normal circumstances.
ii.ii Amino acid condensation: dissolving amino acids protected by N-α-Fmoc, O-benzotriazole-N,N,N′, N′-tetramethyl-uronium-hexafluorophosphate (HBTU), N-hydroxybenzotriazole (HOBt), and N, N-diisopropylethylamine (DIEA) with a molar ratio of 1:0.2˜1.5:0.2˜1.5:1.5˜3 in a small amount of DMF, wherein DIEA is to beadded in the end, the volume to mass ratio of DMF and N-α-Fmoc protected amino acid is 5-10 mL/g; then adding the mixed solution to the resin obtained in step ii.i, wherein the volume to mass ratio of mixed solution and the resin is 4-6 mL/g; the stirring rate is 60-100 rpm; the reaction involves reacting at room temperature for 40-100 minutes. After the reaction, washing the mixture with DMF. The mixture is tested by ninhydrin, the solution is light yellow and the resin is colorless under normal circumstances, resulting in a peptide resin without Fmoc protective group.
ii.iii Extension of peptide chain: according to the peptide sequence, attaching different Fmoc protected amino acids sequentially onto the peptide resin obtained in step ii.ii. The last amino acid is usually protected by tert-butoxycarbonyl (Boc), and eliminating the last step of Fmoc removal. The condensation method for all amino acids is as described above. The peptide resin is obtained.
ii.iv Cyclization of polypeptide: washing the the resin with DCM and methanol (MeOH) alternately, then compressing and extracting to dryness. Under argon protection, adding the resin obtained from step ii.iii to tetratriphenylphosphine palladium (Pd(PPh₃)₄) with a molar weight of 0.55 times the resin and triethylenediamine (DABCO) with a molar weight of 5 times the resin; then adding a mixed solution of chloroform (CHCl₃), acetic acid (HAc), and N-methylmorpholine (NMM) with a volume ratio of 37:2:1 via a syringe, and the volume mass ratio of the mixed solution to the resin is 3-6 mL/g; reacting with slowly stirr at 25° C. for 3-6 hours at a stirring rate of 100-200 rpm, selectively removing the protective groups of allyloxycarbonyl (Alloc) and carboxylallyl ester (OAll). The mixture is tested by ninhydrin, the solution is blue purple and the resin is blue purple under normal circumstances. Then transferring the obtained peptide resin to a synthesizer, washing the resion with DCM and DMF alternately, then adding benzotriazol-1-yl-oxytripyrrolidino-phosphonium hexafluorophosphate (PyBOP) with a molar weight of 3 times the resin and DIEA with a molar weight of 6 times, reacting the cyclization condensation reaction for 3-6 hours and the stirring rate is 60-100 rpm. The mixture is tested by ninhydrin, the solution is light yellow and the resin is colorless under normal circumstances.
iii. Condensing of the resin: washing the cyclic peptide resin with DCM and MeOHse alternately, removing the stirring rod after washing, and extracting for 3-5 hours to dryness.
iv. Cutting and precipitation extraction of peptides: adding a cutting agent (mixed solution of trifluoroacetic acid (TFA), triisopropylsilane (Tis), and water (H₂O) with the volume ratio of 95:2.5:2.5) to the resin obtained in step iii. Reacting the mixture at room temperature for 1.5-4 hours, stirring every 15 minutes. The volume mass ratio of cutting agent to cyclized peptide resin is 10-20 mL/g. Evaporating the cutting liquid to dryness by a rotary evaporator, with a temperature no higher than 40° C. Placing the remaining liquid in a refrigerator for pre-cooling. Then adding pre-cooled ether, stewing to precipitate, and extracting the precipitate with 20% HAc aqueous solution. Then freeze-dring the extracted solution to obtain crude peptides.
v. Purification and analysis of polypeptide: separating and purifiing crude peptides using a reverse phase high-performance liquid chromatography (RP-HPLC) with a semi-preparative column. After separation, collecting the main peak and freeze-dring to obtain pure peptides. The purity is identified by RP-HPLC analysis column, and then the molecular weight of the polypeptide is identified by electrospray ionization mass spectrometry (ESI).

The third aspect of the present invention provides a pharmaceutical composition, comprising:
(a) multi-target cyclopeptide molecule for opioid receptors and neuropeptide FF receptors or a pharmaceutically acceptable salt thereof according to the first aspect of the present invention as active ingredient;
(b) pharmaceutically acceptable carriers and/or excipients.

In another preferred embodiment, the pharmaceutical composition is administered by means selected from the group consisting of oral administration, percutaneous administration, intrathecal administration, intravenous administration, intramuscular administration, local administration, nasal administration, etc.

In another preferred embodiment, the formulation of the pharmaceutical composition is selected from the group consisting of tablets, capsules, sugar coated tablets, granules, oral solutions and syrups, aerosols, nasal sprays, dry powder injections, injections, ointments and patches for skin surface.

In another preferred embodiment, the formulation of the pharmaceutical composition is an injection.

In another preferred embodiment, the drug injection can be used for parenteral administration.

The fourth aspect of the present invention provides a use of multi-target cyclopeptide molecule or a pharmaceutically acceptable salt thereof according to the first aspect of the present invention for the preparation of a drug for alleviating and/or treating various types of pain, including acute pain and pathological pain.

The fifth aspect of the present invention provides a method for analgesia, comprising the steps of administering a safe and effective amount of multi-target cyclopeptide molecule for opioid receptors and neuropeptide FF receptors or a pharmaceutically acceptable salt thereof according to the first aspect of the present invention, and/or a pharmaceutical composition according to the third aspect of the present invention to the subject in need thereof.

In another preferred embodiment, the method is non-therapeutic or therapeutic.

In another preferred embodiment, the method is in vitro method or in vivo method.

In another preferred embodiment, the subject in need thereof is the subject that needs to alleviate and/or treat various types of pain.

In another preferred embodiment, the subject is a mammal or a human.

In another preferred embodiment, the subject is a human.

It should be understood that within the scope of the present invention, the above-described technical features of the present invention and the technical features described in detail below (e.g., examples) may be combined with each other to constitute a new or preferred technical solution. Limited by space, it will not be repeated here.

### DESCRIPTION OF THE DRAWINGS

FIG. 1 shows the time-dose effect curve of the dose-dependent analgesic effect produced by subcutaneous injection of compound 1 in mice.

FIG. 2 shows the time-dose effect curve of the dose-dependent analgesic effect produced by subcutaneous injection of compound 2 in mice.

FIG. 3 shows the time-dose effect curve of the dose-dependent analgesic effect produced by subcutaneous injection of compound 3 in mice.

FIG. 4 shows the time-dose effect curve of the dose-dependent analgesic effect produced by subcutaneous injection of compound 4 in mice.

FIG. 5 shows the time-dose effect curve of the dose-dependent analgesic effect produced by subcutaneous injection of compound 5 in mice.

FIG. 6 shows the time-dose effect curve of the dose-dependent analgesic effect produced by subcutaneous injection of compound 6 in mice.

FIG. 7 shows the time-dose effect curve of the dose-dependent analgesic effect produced by subcutaneous injection of compound 7 in mice.

FIG. 8 shows the time-dose effect curve of the dose-dependent analgesic effect produced by subcutaneous injection of compound 8 in mice.

FIG. 9 shows the time-dose effect curve of the dose-dependent analgesic effect produced by subcutaneous injection of compound 9 in mice.

FIG. 10 shows the time-dose effect curve of the dose-dependent analgesic effect produced by subcutaneous injection of compound 10 in mice.

FIG. 11 shows the time-dose effect curve of the dose-dependent analgesic effect produced by subcutaneous injection of compound 11 in mice.

FIG. 12 shows the time-dose effect curve of the dose-dependent analgesic effect produced by subcutaneous injection of compound 12 in mice.

FIG. 13 shows the time-dose effect curve of the dose-dependent analgesic effect produced by subcutaneous injection of compound 13 in mice.

FIG. 14 shows the time-dose effect curve of the dose-dependent analgesic effect produced by subcutaneous injection of compound 14 in mice.

FIG. 15 shows the time-dose effect curve of the analgesic effect produced by oral injection of parent molecule DN-9 in mice.

FIG. 16 shows the time-dose effect curve of the dose-dependent analgesic effect produced by oral injection of compound 1 in mice.

FIG. 17 shows the time-dose effect curve of the dose-dependent analgesic effect produced by oral injection of compound 11 in mice.

FIG. 18 shows the results of the study on the blood-brain barrier permeability of subcutaneous injection of compound 1 in mice injected with naloxone iodide and naloxone.

FIG. 19 shows the results of the study on the blood-brain barrier permeability of subcutaneous injection of compound 2 in mice injected with naloxone iodide and naloxone.

FIG. 20 shows the results of the study on the blood-brain barrier permeability of subcutaneous injection of compound 3 in mice injected with naloxone iodide and naloxone.

FIG. 21 shows the results of the study on the blood-brain barrier permeability of subcutaneous injection of compound 4 in mice injected with naloxone iodide and naloxone.

FIG. 22 shows the results of the study on the blood-brain barrier permeability of subcutaneous injection of compound 5 in mice injected with naloxone iodide and naloxone.

FIG. 23 shows the results of the study on the blood-brain barrier permeability of subcutaneous injection of compound 6 in mice injected with naloxone iodide and naloxone.

FIG. 24 shows the results of the study on the blood-brain barrier permeability of subcutaneous injection of compound 7 in mice injected with naloxone iodide and naloxone.

FIG. 25 shows the results of the study on the blood-brain barrier permeability of subcutaneous injection of compound 8 in mice injected with naloxone iodide and naloxone.

FIG. 26 shows the results of the study on the blood-brain barrier permeability of subcutaneous injection of compound 9 in mice injected with naloxone iodide and naloxone.

FIG. 27 shows the results of the study on the blood-brain barrier permeability of subcutaneous injection of compound 10 in mice injected with naloxone iodide and naloxone.

FIG. 28 shows the results of the study on the blood-brain barrier permeability of subcutaneous injection of compound 11 in mice injected with naloxone iodide and naloxone.

FIG. 29 shows the results of the study on the blood-brain barrier permeability of subcutaneous injection of compound 12 in mice injected with naloxone iodide and naloxone.

FIG. 30 shows the results of the study on the blood-brain barrier permeability of subcutaneous injection of compound 13 in mice injected with naloxone iodide and naloxone.

FIG. 31 shows the results of the study on the blood-brain barrier permeability of subcutaneous injection of compound 14 in mice injected with naloxone iodide and naloxone.

FIG. 32 shows the results of the study on the blood-brain barrier permeability of oral injection of compound 1 in mice injected with naloxone iodide and naloxone.

FIG. 33 shows the results of the study on the blood-brain barrier permeability of oral injection of compound 11 in mice injected with naloxone iodide and naloxone.

FIG. 34 shows the changes in analgesic effect produced by oral injection of compounds 1 and 11 into mice for eight consecutive days.

FIG. 35 shows the effect of oral injection of compound 1 on gastrointestinal motility in mice.

FIG. 36 shows the effect of oral injection of compound 11 on gastrointestinal motility in mice.

FIG. 37 shows the regulatory effect of oral injection of compounds 1 and 11 on exercise activity in mice.

FIG. 38 shows the regulatory effect of oral injection of compounds 1 and 11 on conditioned site in mice.

FIG. 39 shows the response of mice to naloxone withdrawal after oral injection of compound 1.

FIG. 40 shows the response of mice to naloxone withdrawal after oral injection of compound 11.

### DETAILED DESCRIPTION OF THE INVENTION

Through extensive and in-depth research, the present inventors obtained a series of multi-target cyclopeptide molecules for opioid receptors and neuropeptide FF receptors, or a pharmaceutically acceptable salt thereof for the first time based on the multi-target molecule DN-9 for opioid receptors/NPFF receptors, by introducing amino acid residues D-Dap, D-Dab, Lys, D-Lys, D-Orn, Asp, D-Asp, D-Glu or Glu with amino and carboxyl groups in the side chain and modifying by cyclization for the opioid pharmacophore, introducing Gly, β-Ala, Aib, and Cha for amino acid substitution for the NPFF pharmacophore. Research has shown that the novel cyclopeptide molecule of the present invention or a pharmaceutically acceptable salt thereof overcomes the problems of short analgesic duration and weak peripheral analgesic activity of the parent molecule DN-9, and has efficient peripheral analgesic activity, long analgesic duration, low effective analgesic dose, no tolerance, low addiction, and low constipation. It can be used as a new generation of potential drugs for treating various types of pain. The inventor completed the present invention on this basis.

### Terms

As used herein, the terms “multi-target cyclopeptide molecule for opioid receptors and neuropeptide FF receptors or a pharmaceutically acceptable salt thereof” and “cyclopeptide molecule of the present invention or a pharmaceutically acceptable salt thereof” can be used interchangeably, referring to the amide bond cyclization analogues of multi-target molecule DN-9 for opioid receptor/neuropeptide FF receptors of the present invention or a pharmaceutically acceptable salt thereof. The cyclopeptide moleculeis obtained by using the multi-target molecule DN-9 for opioid receptor/neuropeptide FF receptors as a chemical template, introducing amino acid residues D-Dap, D-Dab, Lys, D-Lys, D-Orn, Asp, D-Asp, D-Glu or Glu with amino and carboxyl groups in the side chain and cyclization modification by amide bond for opioid pharmacophore, introducing Gly, β-Ala, Aib, and Cha for amino acid substitution for the NPFF pharmacophore. Among them, the sequence of the the multi-target parent peptide DN-9 molecule is as follows:
Tyr-D-Ala-Gly-NMe-Phe-Gly-Pro-Gln-Arg-Phe-NH₂.

In another preferred embodiment, the specific sequence of the cyclopeptide molecule is shown in Table 1:

TABLE 1
Amino acid sequences of multi-target
cyclopeptide molecules for opioid receptors
and neuropeptide FF receptors

| Compounds | Amino acid sequences |
|---|---|
| Compound 1 | Tyr-c[D-Lys-Gly- |
| | NMe-Phe-Asp]-Pro-Gln-Arg-Phe-NH₂ |
| Compound 2 | Tyr-c[D-Lys-Gly-NMe-Phe-Glu]- |
| | Pro-Gln-Arg-Phe-NH₂ |
| Compound 3 | Tyr-c[Lys-Gly-NMe-Phe-Asp]- |
| | Pro-Gln-Arg-Phe-NH₂ |
| Compound 4 | Tyr-c[Lys-Gly-NMe-Phe-D-Asp]- |
| | Pro-Gln-Arg-Phe-NH₂ |
| Compound 5 | Tyr-c[D-Lys-Gly-NMe-Phe-D- |
| | Asp]-Pro-Gln-Arg-Phe-NH₂ |
| Compound 6 | Tyr-c[D-Asp-Gly-NMe-Phe-Lys]- |
| | Pro-Gln-Arg-Phe-NH₂ |
| Compound 7 | Tyr-c[D-Glu-Gly-NMe-Phe-Lys]- |
| | Pro-Gln-Arg-Phe-NH₂ |
| Compound 8 | Tyr-c[D-Orn-Gly-NMe-Phe-Asp]- |
| | Pro-Gln-Arg-Phe-NH₂ |
| Compound 9 | Tyr-c[D-Dab-Gly-NMe-Phe- |
| | Asp]-Pro-Gln-Arg-Phe-NH₂ |
| Compound 10 | Tyr-c[D-Dap-Gly-NMe-Phe- |
| | Asp]-Pro-Gln-Arg-Phe-NH₂ |
| Compound 11 | Tyr-c[D-Orn-Gly-NMe-Phe- |
| | Glu]-Pro-Gln-Arg-Phe-NH₂ |
| Compound 12 | Tyr-c[D-Lys-Gly-NMe-Phe- |
| | Asp]-Gly-Gln-Arg-Phe-NH₂ |
| Compound 13 | Tyr-c[D-Lys-Gly-NMe-Phe- |
| | Asp]-Pro-β-Ala-Arg-Cha-NH₂ |
| Compound 14 | Tyr-c[D-Lys-Gly-NMe-Phe- |
| | Asp]-Pro-Aib-Arg-Cha-NH₂ |

As used herein, the terms “NMe-Phe”, “N-Me-Phe”, or “NMePhe” can be used interchangeable and refer to N^{α}-Methylphenylalanine, its structural formula is as follows:

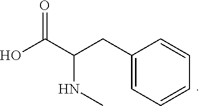

As used herein, the conventional three letter is used to represent natural amino acids, and the recognized three letter is used to represent other amino acids, such as NMe-Phe (N^{α}-Methylphenylalanine), D-Lys (D-lysine), D-Orn (D-ornithine), D-Dab (D-2,4-diaminobutyric acid), D-Dap (D-2,4-diaminopropionic acid), D-Asp (D-aspartic acid), D-Glu (D-glutamic acid) β-Ala (β-alanine), Aib (aminoisobutyric acid), Cha(β-cyclohexyl-L-alanine). In addition, the amino acids described in the present invention are L-type amino acids, except for those specifically indicated that “D-” represents D-type amino acids.

As used herein, the term “pharmaceutically acceptable salt” refers to a salt of the cyclopeptide molecule of the present invention that can retain the biological efficacy of the cyclopeptide molecule of the present invention without any other side effects, and is synthesized with non-toxic acids or bases.

### Preparation Method

The multi-target cyclopeptide molecule for opioid receptors and neuropeptide FF receptors of the present invention or pharmaceutically acceptable salt thereof uses DN-9 as a chemical template, and optimizes the structure of opioid peptides pharmacophore and neuropeptide FF pharmacophore using peptide chemical strategies such as amino acid substitution and cyclization modification by amide bond. The specific process steps include:
i. Resin pretreatment: Rink-Amide-MBHA resin is swelled in DCM, extracted to dryness, the resin is washed with DMF, and tested using ninhydrin.
ii. Performing amino acid condensation in turn:
ii.i Removal of Fmoc protecting group: A mixed solution of DBU, piperidine, and DMF with a volume ratio of 1:1:98 is added to the resin obtained in step i. The mixture is stirred for carrying out the reaction. DMF is added to the mixture for washing and the mixture is tested by ninhydrin.
ii.ii Amino acid condensation: Fmoc-Aa, HBTU, HOBt, and DIEA are dissolved in DMF with a molar ratio of 1:0.2˜1.5:0.2˜1.5:1.5˜3, and then the mixed solution is added to the resin obtained in step ii.i. The mixture is washed with DMF and tested by ninhydrin to obtain peptide resin without Fmoc protective groups.
ii.iii Extension of peptide chain: According to the peptide sequence, different Fmoc protected amino acids are sequentially attached, and the last amino acid is protected by Boc. The condensation method for all amino acids is as described above to obtain a peptide resin.
ii.iv Cyclization of peptides: The peptide resin is washed alternately with DCM and MeOH, the mixture is compressed and extracted to dryness; under argon protection, Pd(PPh₃)₄ and DABCO were added to the peptide resin obtained in step ii.iii, and then a mixture of CHCl₃, HAc, and NMM was added using a syringe to selectively remove Alloc and OAll protective groups; the mixture is tested by ninhydrin, PyBOP and DIEA are added after the mixture is washed, then cyclization condensation reaction is carried out for 3-6 hours at a stirring rate of 60-100 rpm, and the mixture is tested using ninhydrin to obtain cyclized peptide resin;
iii. Compression of the resin: Cyclized peptide resin is wahsed alternately with DCM and MeOH, and extracted to dryness;
iv. Cutting and precipitation extraction of peptides: Cutting agent (mixed solution of TFA, Tis, and H₂O with a volume ratio of 95:2.5:2.5) is added to the peptide resin obtained in step iii, and the reaction is carried out at room temperature for 1.5-4 h; the mixture is rotary evaporated, pre-cooled ether is added to precipitate, the precipitate is extracted with 20% HAc aqueous solution, and the extracted solution is freeze-dried to obtain crude peptides;
v. Purification and analysis of peptides: Crude peptides are separated and purified using a reverse phase high-performance liquid chromatography (RP-HPLC) with a sem-preparative column. After separation, the main peak is collected and freeze-dried to obtain pure peptides. Then the molecular weight of the polypeptide is identified by electrospray ionization (ESI) mass spectrometry.

### Pharmaceutical Composition and Administration

The present invention also provides a pharmaceutical composition comprising a safe and effective amount of cyclopeptide molecule or a pharmaceutically acceptable salt thereof, as well as pharmaceutically acceptable carriers.

The cyclopeptide molecule of the present invention or a pharmaceutically acceptable salt thereof and pharmaceutical composition can be used for preparing analgesic drugs. The cyclopeptide molecule of the present invention or a pharmaceutically acceptable salt thereof and pharmaceutical composition can be used as analgesic drug.

In another preferred embodiment, it can be used to prepare an analgesic drug with high efficiency and low side effects.

In another preferred embodiment, it can be used to alleviate and treat various types of pain, including acute pain and pathological pain.

The “safe and effective amount” refers to the amount of the cyclopeptide molecule of the present invention or a pharmaceutically acceptable salt thereof that is sufficient to produce significant analgesic effect without causing serious side effects.

The pharmaceutical composition can be in any suitable form, depending on the method of administration required by the patient. It can be provided in the form of a unit dosage form, usually placed in a sealed container, and can be provided as a part of kit. This type of kit usually (but not necessarily) contains instructions for use. It can include multiple unit dosage forms.

The administration methods of the pharmaceutical composition are not particularly limited, and representative administration methods include (but are not limited to) oral administration, percutaneous administration, intrathecal administration, intravenous administration, intramuscular administration, local administration, nasal administration, etc.

According to the administration methods used, the pharmaceutical composition of the present invention can be prepared as various suitable dosage forms. Examples of suitable dosage forms include sterile solutions that can be used for injection and dry powder injections, tablets, capsules, sugar coated tablets, granules, oral solutions and syrups, aerosols, nasal sprays, as well as ointments and patches for skin surfaces.

The pharmaceutical composition containing the cyclopeptide molecule of the present invention or a pharmaceutically acceptable salt thereof can be prepared into solutions or freeze-dried powders for parenteral administration. Before use, appropriate solvents or other pharmaceutically acceptable carriers can be added to reconfigure the powder. The solution formula is generally a buffer, isotonic solution, or aqueous solution.

The pharmaceutical composition of the present invention can be administered separately or in combination with other analgesic drugs.

When using the pharmaceutical composition, a safe and effective amount of the peptide of the present invention or a pharmaceutically acceptable salt thereof is administered to mammals (such as humans) in need of treatment. The dosage of the pharmaceutical composition of the present invention can vary within a large range, and can be easily determined by those skilled in the art depending on objective factors such as the type of disease, severity of the condition, patient weight, dosage form, route of administration.

Compared with prior art, the main advantages of the present invention include:
(1) The subcutaneous analgesic activity and oral analgesic activity of the cyclopeptide molecule of the present invention or a pharmaceutically acceptable salt thereof are significantly improved compared to the parent molecule DN-9, the analgesic ED₅₀ (half effective dose) is reduced by hundreds of times, preferably up to 1000-10000 times when subcutaneous injected and analgesic activity is also improved compared to the parent molecule when orally administered.
(2) The cyclopeptide molecule of the present invention or a pharmaceutically acceptable salt thereof has a long actuation duration of about 240 minutes.
(3) The cyclopeptide molecule of the present invention or a pharmaceutically acceptable salt thereof cannot penetrate the blood-brain barrier, has no analgesic tolerance, constipation, or addictive side effects.

The present invention will be further explained below in combination with specific examples. It should be understood that these examples are only used to illustrate the present invention and not to limit the scope of the present invention. In the following examples, the test methods without specific conditions are usually in accordance with conventional conditions or the conditions recommended by the manufacturer. Unless otherwise specified, percentages and parts are calculated by weight.

Unless otherwise defined, all professional and scientific terms used herein have the same meanings as those familiar to those skilled in the art. In addition, any methods and materials similar or equal to the recorded content can be applied to the methods of the present invention. The preferred implementation methods and materials described herein are only for demonstration purposes.

The instruments and main experimental materials used are as follows:
Experimental instruments: The solid-phase peptide synthesizer was independently designed by the inventor. The rotary evaporator RE-5298A was purchased from Shanghai Yarong, the freeze-drying machine was purchased from VIRTIS in the United States, the mass spectrometer ESI-Q-TOF maXis-4G, Bruker Daltonics was purchased from Dalton in Germany, the circulating water pump SHB-III was purchased from Zhengzhou Great Wall. The high-performance liquid chromatography (RP-HPLC) was Delta 600 from Waters, wherein the analytical column: XBridge™ BEH 130 Prep C18, 4.6 mm×250 mm; Preparation column: XBridge™ BEH 130 Prep C18, 19 mm×250 mm.
Experimental reagents: The resin is Rink-Amide-MBHA Resin (with a substitution value S of 0.4 mmol/g), purchased from Tianjin Nankai Hecheng Company. N-α-Fmoc protected amino acids (Fmoc-Aa), O-benzotriazole-N,N,N′,N′-tetramethyl-uronium-hexafluorophosphate (HBTU), and N-hydroxybenzotriazole (HOBt) were purchased from Shanghai Jier Biochemical Co., Ltd.. N,N-diisopropylethylamine (DIEA) was purchased from Beijing J&K, and 1,8-diazabicycloundecano-7-ene (DBU) was purchased from Shanghai MERYER Company. Tetratriphenylphosphine palladium (Pd(PPh₃)₄), triethylenediamine (DABCO), and triisopropylsilane (Tis) were purchased from Shanghai Energy, and ninhydrin was a product of Shanghai Reagent Factory III. Dichloromethane (DCM), N,N-dimethylformamide (DMF), hexahydropyridine (piperidine), methanol (MeOH), and pyridine were purchased from Tianjin Reagent Factory II, and trifluoroacetic acid (TFA) and phenol were all products of Tianjin Reagent Factory. The above organic reagents have undergone redistillation treatment before use.

### Example 1: Synthesis of Compound 1

(1) Resin pretreatment: 1 g of Rink-Amide-MBHA resin (with a substitution value of 0.4 mmol/g) was weighed, and 10 mL of DCM was added, the mixture was stirred at 80 rpm for swelling, stirred and reacted for 30 minutes, extracted to dryness, and the resin was rinsed with DMF three times, each time for 3 minutes.
(2) Removal of Fmoc protecting groups: To the resin 10 mL of mixed solution of DBU, piperidine, and DMF in a volume ratio of 1:1:98 was added; then stirred at 80 rpm for 5 minutes and repeated twice. After extracting to dryness, the above mixed solution was added again and then stirred at 80 rpm for 10 minutes. Finally, the mixture was washed 4 times by adding DMF, each time for 3 minutes.
(3) Ninhydrin test: The ninhydrin test reagent is a mixture of solutions of phenol: pyridine: ninhydrin with a volume ratio of 1:2:1, wherein phenol solution was prepared by adding 20 g of phenol to 5 mL of anhydrous ethanol, pyridine solution was prepared by adding 0.05 mL of KCN (0.001 M) into 2.5 mL of pyridine, and ninhydrin solution was prepared by adding 0.5 g of ninhydrin into 10 mL of anhydrous ethanol. Among them, phenol, pyridine, and anhydrous ethanol are all redistilled. Ninhydrin test, the solution is blue purple and the resin is blue purple under normal circumstances.
(4) Amino acid condensation: Fmoc-Phe-OH, HOBt, and HBTU were weighed in a molar ratio of 1:1:1, and dissolved in 5 mL of DMF, wherein DIEA was finally added. After stirred well, the mixture was added to the resin that had been removed the Fmoc protective group in step (2). Under argon protection, the reaction was carried out for 60 minutes at room temperature at a stirring rate of 80 rpm, and the solvent was extracted to dryness. After the reaction is completed, the mixture was tested by ninhydrin according to the method in step (3). If the solution was light yellow and the resin was colorless, it indicated that amino acids had condensed onto the resin. Then, the Fmoc protective groups were removed according to the step (2), and the resulting mixture was tested by ninhydrin again according to the step (3). If the solution and resin were both dark blue, it indicated that the Fmoc protective groups had been completely removed, resulting in a resin peptide without Fmoc protective groups.
(5) Extension of peptide chain: Fmoc-Arg-(pbf)-OH, Fmoc-Gln-(Trt)-OH, Fmoc-Pro-OH, Fmoc-Asp-(OAll)-OH, Fmoc-NMe-Phe-OH, Fmoc-Gly-OH, Fmoc-D-Lys-(Alloc)-OH, and Boc-Tyr-(tBu)-OH were sequentially condensed onto the peptide resin obtained in step (4) according to the method in step (2) to (4) to obtain peptide resin Boc-Tyr-(tBu)-D-Lys-(Alloc)-Gly-NMe-Phe-Asp-(OAll)-Pro-Gln-(Trt)-Arg-(Pbf)-Phe-Resin.
(6) Cyclization of polypeptide: The obtained peptide resin Boc-Tyr-(tBu)-D-Lys-(Alloc)-Gly-NMe-Phe-Asp-(OAll)-Pro-Gln-(Trt)-Arg-(Pbf)-Phe-Resin was sequentially washed with DCM (2×3 min), MeOH (1×3 minutes), DCM (1×3 min), and MeOH (2×3 minutes) alternately, then condensed, and drain for 4 hours to dryness. Then the peptide resin was transferred to a round bottom flask, 255 mg of Pd(PPh₃)₄ and 225 mg of DABCO were added, the atmosphere was protected by argon, and then 5 mL of the mixed solution of CHCl₃, HAc, and NMM with a volume ratio of 37:2:1 was added using a syringe. Under argon protection, the mixture was stirred at 150 rpm and reaction was carried out at 25° C. for 4 hours to selectively remove amino Alloc and carboxyl OAll. The resulting mixture was tested by ninhydrin the solution is blue purple and the resin is blue purple under normal circumstances. Then 625 mg of PyBOP and 396 μL of DIEA were added, the mixture was stirred at 80 rpm and the reaction was carried out for 4 hours. The resulting mixture was tested by ninhydrin, the solution was light yellow, and the resin was colorless to obtain cyclized peptide Boc-Tyr(tBu)-c^{[2,5]}[D-Lys(Alloc)-Gly-NMe-Phe-Asp(OAll)]-Pro-Gln(Trt)-Arg(Pbf)-Phe-Resin.
(7) Condensing and drying of peptide chains: DCM (2×3 min), MeOH (1×3 minutes), DCM (1×3 min), and MeOH (2×3 minutes) were used to wash the resin alternately, then the stirring rod was removed and the resin was extracted for 4 hours to dryness.
(8) Cutting of peptide chains: 15 mL of cutting agent (TFA: H₂O: Tis=95:2.5:2.5) was added into dried peptide resin Boc-Tyr(tBu)-c^{[2,5]}[D-Lys(Alloc)-Gly-NMe-Phe-Asp(OAll)]-Pro-Gln(Trt)-Arg(Pbf)-Phe-Resin. The reaction was carried out for 3 hours. The mixture was stirred for 1 minute every 15 minutes at a stirring rate of 50 rpm. The filtrate was fully spin-dried under reduced pressure at not exceeding 40° C. Cooled ether was added and the mixture was shaken and mixed well. The crude peptide was fully precipitated in the form of white precipitate. The crude peptide from ether was extracted with 20% HAc aqueous solution. Finally, the extracted peptide aqueous solution was freeze-dried to obtain 324 mg of white crude peptide solid powder, with a crude peptide yield of 70%.
(9) Purification of crude peptide: The above crude peptide compound was separated and purified using reverse phase high-performance liquid chromatography (RP-HPLC) C18 column(XBridge™ BEH 130 Prep C18, 19 mm×250 mm), and acetonitrile (containing 0.1% TFA) and water (containing 0.1% TFA) were used. After separation, the main peak sample was collected to obtain a purified compound 1 sample with a loading amount of 50 mg. 15 mg of white pure peptide solid powder was obtained by freeze-drying, with a pure peptide yield of 30%. The results of mass spectrometry and chromatography analysis were shown in Table 2.

### Example 2. Synthesis of Compound 2

According to the same method as Example 1, the cyclopeptide Tyr-c^{[2,5]}[D-Lys-Gly-NMe-Phe-Glu]-Pro-Gln-Arg-Phe-NH₂ was obtained as a white solid powder, the crude peptide was 300 mg with the yield of 64%, after refinement, the yield of a pure peptide was 24%. The results of mass spectrometry and chromatography analysis were shown in Table 2.

### Example 3. Synthesis of Compound 3

According to the same method as Example 1, the cyclopeptide Tyr-c^{[2,5]}[Lys-Gly-NMe-Phe-Asp]-Pro-Gln-Arg-Phe-NH₂ was obtained as a white solid powder, the crude peptide was 320 mg with the yield of 69%, after refinement, the yield of a pure peptide was 20%. The results of mass spectrometry and chromatography analysis were shown in Table 2.

### Example 4. Synthesis of Compound 4

According to the same method as Example 1, the cyclopeptide Tyr-c^{[2,5]}[Lys-Gly-NMe-Phe-D-Asp]-Pro-Gln-Arg-Phe-NH₂ was obtained as a white solid powder, the crude peptide was 325 mg with the yield of 71%, after refinement, the yield of a pure peptide was 26%. The results of mass spectrometry and chromatography analysis were shown in Table 2.

### Example 5. Synthesis of Compound 5

According to the same method as Example 1, the cyclopeptide Tyr-c^{[2,5]}[D-Lys-Gly-NMe-Phe-D-Asp]-Pro-Gln-Arg-Phe-NH₂ was obtained as a white solid powder, the crude peptide was 350 mg with the yield of 76%, after refinement, the yield of a pure peptide was 30%. The results of mass spectrometry and chromatography analysis were shown in Table 2.

### Example 6. Synthesis of Compound 6

According to the same method as Example 1, the cyclopeptide Tyr-c^{[2,5]}[D-Asp-Gly-NMe-Phe-Lys]-Pro-Gln-Arg-Phe-NH₂ was obtained as a white solid powder, the crude peptide was 330 mg with the yield of 72%, after refinement, the yield of a pure peptide was 60%. The results of mass spectrometry and chromatography analysis were shown in Table 2.

### Example 7. Synthesis of Compound 7

According to the same method as Example 1, the cyclopeptide Tyr-c^{[2,5}][D-Glu-Gly-NMe-Phe-Lys]-Pro-Gln-Arg-Phe-NH₂ was obtained as a white solid powder, the crude peptide was 443 mg with the yield of 95%, after refinement, the yield of a pure peptide was 26%. The results of mass spectrometry and chromatography analysis were shown in Table 2.

### Example 8. Synthesis of Compound 8

According to the same method as Example 1, the cyclopeptide Tyr-c^{[2,5]}[D-Orn-Gly-NMe-Phe-Asp]-Pro-Gln-Arg-Phe-NH₂ was obtained as a white solid powder, the crude peptide was 285 mg with the yield of 63%, after refinement, the yield of a pure peptide was 20%. The results of mass spectrometry and chromatography analysis were shown in Table 2.

### Example 9. Synthesis of Compound 9

According to the same method as Example 1, the cyclopeptide Tyr-c^{[2,5}][D-Dab-Gly-NMe-Phe-Asp]-Pro-Gln-Arg-Phe-NH₂ was obtained as a white solid powder, the crude peptide was 290 mg with the yield of 65%, after refinement, the yield of a pure peptide was 40%. The results of mass spectrometry and chromatography analysis were shown in Table 2.

### Example 10. Synthesis of Compound 10

According to the same method as Example 1, the cyclopeptide Tyr-c^{[2,5]}[D-Dap-Gly-NMe-Phe-Asp]-Pro-Gln-Arg-Phe-NH₂ was obtained as a white solid powder, the crude peptide was 300 mg with the yield of 68%, after refinement, the yield of a pure peptide was 24%. The results of mass spectrometry and chromatography analysis were shown in Table 2.

### Example 11. Synthesis of Compound 11

According to the same method as Example 1, the cyclopeptide Tyr-c^{[2,5]}[D-Orn-Gly-NMe-Phe-Glu]-Pro-Gln-Arg-Phe-NH₂ was obtained as a white solid powder, the crude peptide was 330 mg with the yield of 72%, after refinement, the yield of a pure peptide was 38%. The results of mass spectrometry and chromatography analysis were shown in Table 2.

### Example 12. Synthesis of Compound 12

According to the same method as Example 1, the cyclopeptide Tyr-c^{[2,5]}[D-Lys-Gly-NMe-Phe-Asp]-Gly-Gln-Arg-Phe-NH₂ was obtained as a white solid powder, the crude peptide was 307 mg with the yield of 69%, after refinement, the yield of a pure peptide was 50%. The results of mass spectrometry and chromatography analysis were shown in Table 2.

### Example 13. Synthesis of Compound 13

According to the same method as Example 1, the cyclopeptide Tyr-c^{[2,5]}[D-Lys-Gly-NMe-Phe-Asp]-Pro-P-Ala-Arg-Cha-NH₂ was obtained as a white solid powder, the crude peptide was 329 mg with the yield of 74%, after refinement, the yield of a pure peptide was 28%. The results of mass spectrometry and chromatography analysis were shown in Table 2.

### Example 14. Synthesis of Compound 14

According to the same method as Example 1, the cyclopeptide Tyr-c^{[2,5]}[D-Lys-Gly-NMe-Phe-Asp]-Pro-Aib-Arg-Cha-NH₂ was obtained as a white solid powder, the crude peptide was 423 mg with the yield of 93%, after refinement, the yield of a pure peptide was 24%. The results of mass spectrometry and chromatography analysis were shown in Table 2.

Based on the above synthesis steps, the present invention synthesized multi-target cyclopeptide molecule for opioid receptors and neuropeptide FF receptors including those as listed in Table 1, and the chemical characterization results were shown in Table 2.

TABLE 2
Chemical characterization of the cyclopeptide molecule of the present invention

| | | | | | High resolution mass | | |
|---|---|---|---|---|---|---|---|
| | System 1 | | System 2 | | spectrometry m/z (M + H⁺) | | Melting |
| | t_{R} | purity | t_{R} | purity | calculation | detection | point |
| Compound | (min) | (%) | (min) | (%) | value | value | MP (° C.) |
| Compound 1 | 17.31 | 98.2 | 16.08 | 99.8 | 1152.6 | 1152.6 | 185-187 |
| Compound 2 | 17.14 | 98.9 | 15.89 | 100.0 | 1166.6 | 1166.6 | 177-179 |
| Compound 3 | 17.29 | 98.3 | 14.92 | 99.6 | 1152.6 | 1152.9 | 180-182 |
| Compound 4 | 15.41 | 97.3 | 14.90 | 97.6 | 1152.6 | 1152.6 | 184-186 |
| Compound 5 | 17.44 | 97.2 | 16.06 | 96.4 | 1152.6 | 1152.6 | 182-184 |
| Compound 6 | 15.51 | 95.3 | 14.35 | 98.3 | 1152.6 | 1152.6 | 163-165 |
| Compound 7 | 18.57 | 100.0 | 17.17 | 100.0 | 1166.6 | 1166.6 | 182-184 |
| Compound 8 | 17.38 | 97.5 | 15.81 | 97.8 | 1138.6 | 1138.6 | 177-179 |
| Compound 9 | 17.61 | 98.8 | 15.87 | 98.0 | 1124.6 | 1124.6 | 184-186 |
| Compound 10 | 14.40 | 97.8 | 13.15 | 100.0 | 1110.5 | 1110.6 | 178-180 |
| Compound 11 | 16.06 | 95.9 | 14.89 | 95.6 | 1152.6 | 1152.6 | 156-158 |
| Compound 12 | 11.21 | 95.5 | 11.23 | 98.7 | 1112.6 | 1112.6 | 161-163 |
| Compound 13 | 15.21 | 98.0 | 14.74 | 99.0 | 1101.6 | 1101.6 | 175-177 |
| Compound 14 | 19.71 | 98.5 | 17.64 | 98.2 | 1115.6 | 1115.6 | 196-198 |

Note:
System 1: Gradient elution system 1 was 10-80% acetonitrile/water (0.1% TFA) (completed in 30 minutes), flow rate: 1 mL/min, detection wavelength: 220 nm, analysis chromatographic column: XBridge ™ BEH 130 Prep C18, 4.6 mm × 250 mm. System 2: Gradient elution system 2 was 10-100% acetonitrile/water (0.1% TFA) (completed in 30 minutes), flow rate: 1 mL/min, detection wavelength: 220 nm, analytical column: XBridge ™ BEH 130 Prep C18, 4.6 mm × 250 mm.

### Example 15. In Vitro Functional Activity Assay of Opioid Receptors and NPFF Receptors

The agonistic activity of the cyclopeptide molecule of the present invention against Mu-opioid receptor, Delta-opioid receptor, Kappa-opioid receptor, NPFF₁ receptor and NPFF₂ receptor was evaluated by detecting the regulation of the cyclopeptide molecule of the present invention on the intracellular accumulation of cyclic adenosine monophosphate (cAMP) induced by Forskolin in HEK293 cells stably expressing these five receptors. The experimental method was: the cells were seeded in 24 well plates with 120000 cells per well, and were cultured for more than 20 hours. At the beginning of the experiment, the culture medium in the culture dish was suck out, and then 500 μL of preheated serum-free medium containing 1 mM of IBMX was added, the mixture was incubated at 37° C. for 10 minutes. Then each 10 μL of the drug to be tested and 10 μM of forskolin (final concentration) were added to each well. The mixture was incubated at 37° C. for 30 minutes. After incubation, all the liquid was suck out from the culture dish, 500 μL of 0.2 N hydrochloric acid was added to each well, the mixture was incubated at room temperature for 30 minutes to promote cell lysis. After lysis was complete, NaOH was added to neutralize the hydrochloric acid solution used for lysis. Then all the liquid was suck out from the culture dish into centrifuge tubes, the liquid was centrifuged at 12000 rpm for 2 minutes. 50 μL was taken to a clean centrifuge tube, 100 μL of 60 μg/μL PKA was added, and 100 μL of TE cAMP buffer was added to the blank control group (B). Another 50 μL of 0.5 μ Ci[³H]cAMP was added to each of the above centrifuge tubes, the mixture was quickly mixed and incubated at 4° C. for more than 2 hours. After incubation, another 100 μL of activated carbon suspension was added to each tube, the mixture was vortex agitated evenly, placed in an ice bath for 1 minute, centrifuged at 5000 rpm for 4 minutes. 200 μL of supernatant after centrifugation in each tube was suck out to a 24 well plate, with an additional 700 μL of scintillator liquid added per well, then the 24 well plate was sealed with adhesive film, placed for 3 hours, then placed on the scintillation instrument for measurement.

The inhibitory effect of cAMP was represented by the percentage (% control) of drug inhibition of intracellular cAMP accumulation induced by Forskolin, wherein % control=(cAMP content when treated with Forskolin−cAMP content when Forskolin co-treated with the test drug)/(cAMP content when treated with Forskolin−cAMP content when treated with solvent). The related % control data was represented by mean±S.E.M. The dose-effect relationship of the drug was statistically analyzed using a nonlinear regression model, and the IC₅₀ values of the multi-target cyclopeptide molecule on inhibition of Forskolin induced intracellular cAMP accumulation were calculated using the statistical software GraphPad Prism version 5.0. The experimental results are shown in Tables 3 and 4.

TABLE 3
cAMP functional experiments of the cyclopeptide molecule
of the present invention on opioid receptors

| | IC₅₀value (nM) (95% confidence interval) | | |
|---|---|---|---|
| | Mu-opioid | Kappa-opioid | Delta-opioid |
| Compound | receptor | receptor | receptor |
| Compound 1 | 93.5 (26.1 to 334.3) | 39.0 (12.1 to 124.9) | 549.3 (191.5 to 1576) |
| Compound 2 | 48.8 (13.5 to 176.4) | 29.5 (17.4 to 49.9) | 36.4 (8.1 to 163.0) |
| Compound 3 | 70.7 (17.2 to 290.7) | 320.0 (117.7 to 869.8) | 28.2 (6.1 to 129.6) |
| Compound 4 | 166.5 (45.2 to 612.9) | — | 922.6 (614.4 to 1385) |
| Compound 5 | 586.1 (155.2 to 2214) | — | 345.1 (97.3 to 1224) |
| Compound 6 | 66.3 (23.2 to 189.5) | 58.0 (15.5 to 217.1) | 74.2 (18.6 to 296.4) |
| Compound 8 | 168.3 (40.7 to 696.5) | 138.9 (41.4 to 466.3) | 659.4 (289.8 to 1501) |
| Compound 9 | 37.9 (10.1 to142.6) | 71.2 (17.4 to 290.9) | 95.1 (34.5 to 262.4) |
| Compound 10 | 237.7 (55.1 to 1026) | 136.4 (31.6 to 589.1) | 373.9 (115.7 to 1208) |
| Compound 11 | 84.0 (41.3 to 170.8) | 109.2 (57.6 to 207.0) | 1558 (793.5 to 3061) |
| Compound 12 | 82.6 (21.5 to 317.4) | 97.4 (34.6 to 273.8) | 45.2 (12.3 to 165.4) |

TABLE 4
cAMP functional experiments of the cyclopeptide molecule
of the present invention on NPFF receptors

IC₅₀value (nM) (95% confidence interval)

| Compound | NPFF1receptor | NPFF2receptor |
|---|---|---|
| Compound 1 | 103.9 (25.8 to 418.3) | 49.8 (15.2 to 163.4) |
| Compound 2 | 189.0 (49.1 to 727.8) | 83.1 (21.4 to 323.0) |
| Compound 3 | 28.9 (14.3 to 58.4) | 111.9 (44.4 to 282.2) |
| Compound 4 | 103.9 (25.8 to 418.3) | 81.5 (17.4 to 381.5) |
| Compound 5 | 528.2 (189.3 to 1474) | 189.6 (40.8 to 881.6) |
| Compound 6 | 221.9 (61.8 to 797.4) | 302.8 (80.6 to 1137) |
| Compound 8 | 438.6 (107.1 to 1795) | 132.9 (45.0 to 392.4) |
| Compound 9 | 180.1 (87.3 to 371.9) | 264.8 (58.1 to 1207) |
| Compound 10 | 90.1 (25.4 to 319.1) | 349.1 (91.8 to 1328) |
| Compound 11 | 42.9 (10.2 to 180.4) | 571.9 (197.0 to 1749) |
| Compound 12 | 58.6 (15.3 to 224.7) | 93.0 (32.2 to 268.1) |

As shown in Table 3, in the HEK293 cell line stably expressing Mu- and Delta-opioid receptors, compounds 1-6 and 8-12 dose-dependently inhibited cAMP accumulation induced by forskolin, indicating that these compounds exhibit agonistic activity for Mu- and Delta-opioid receptors. In the HEK293 cell line stably expressing Kappa-opioid receptor, compounds 1-3, 6, and 8-12 dose-dependently inhibited cAMP accumulation induced by forskolin, indicating that compounds 1-3, 6, and 8-12 exhibit agonistic activity for Kappa-opioid receptor. Moreover, as shown in Table 4, in HEK293 cell lines stably expressing NPFF₁ and NPFF₂ receptors, compounds 1-6 and 8-12 dose-dependently inhibited cAMP accumulation induced by forskolin, indicating that these compounds also exhibit both agonist activity for NPFF₁ and NPFF₂ receptors. In summary, compounds 1-6 and 8-12 can simultaneously activate opioid and NPFF receptors, exhibiting a class of multi-target agonists for opioid receptors and NPFF receptors.

### Example 16. In Vivo Analgesic Activity Assay

Two administration methods were used: peripheral subcutaneous administration and oral administration. Subsequently, the analgesic activity of the drug was studied using an acute pain model of photothermal tail flick in mice.

Subcutaneous administration on the back was chosen for subcutaneous administration (s.c.). A sterile 1 mL syringe was used and the drug with the volume of 0.1 mL/10 g was injected for administration. The back skin was grasped with the right hand, the needle was tilted in, and the drug was injected subcutaneously into the mouse's back. After the needle was inserted, shook it left and right, and observe to confirm that the needle had indeed entered the subcutaneous area to prevent the needle from puncturing the skin and leaking the drug.

1 mL sterile syringe was used for oral administration (p.o.), the pinhead was replaced with a mouse gastric lavage needle, and 0.1 μL/10 g of the drug was orally administered. The skin on the neck and back of the mouse was grasped with the left hand with the abdomen facing upwards. Noted that the mouse's entire body was fixed to vertical, which was beneficial for oral gavage. A gastric lavage needle (No. 12 needle and 1 mL syringe) was held in the right hand and was inserted to the esophagus along the upper jaw which is pressed the tongue tightly from the corner of the mouse's mouth. Gastric lavage fluid can be injected when the needle inserted 2.5 cm. The inserting length of the needle should be determined through practice in advance, as there is a sense of falling into a hole after the needle is inserted. If the injection position was appropriate, it should be very smooth. Otherwise, it indicated that the injection was not appropriate, indicating that the gavage needle may be inserted into the mouse's trachea. This can cause immediate death of the mouse after gavage.

The photothermal tail flick experiment is based on the experimental method summarized by D'Amour and Smith to optimize the experimental parameters. The experiment selected Kunming male mice with body weigh 21±2 g, and ambient temperature controlled at 22±2° C. The experimental mice were allowed to drink water freely and were moved from the breeding room to the experimental area for adaptation for 30 minutes before starting the experiment. Then the mice were grab with the right hand and the tail hang freely. Then the mice's tail were placed on a radiation light source, 2-3 mm away from the mice tail. The intensity of radiation heat was adjusted to the tail flick time of mice being 3-5 s, which is the basic pain threshold of photothermal tail flick. The irradiation time of the mice tail should not exceed 10 s to prevent scalding of the mice tail, which was the maximum latency for photothermal tail flick. After administration, time points of 10, 20, 30, 45, 60, 90, 120, 180, 240, 300, 360, and 420 minutes were selected to measure the tail flick latency of mice after administration.

The analgesic effect of drugs was generally evaluated using the maximum analgesic effect MPE (%), MPE (%)=100×[(pain threshold after administration−basic pain threshold)/(10 seconds−basic pain threshold)]. 50% effective dose (ED₅₀) refers to the corresponding drug dose that causes a 50% effect. ED₅₀ and 95% confidence interval were calculated using MPE (%) and the drug concentration that achieves the maximum analgesic effect MPE (%) by the statistical software GraphPad Prism 5.0. The difference in analgesic effects was statistically analyzed using one-way ANOVA (Dunnett test), *P<0.05, **P<0.01, and ***P<0.001 indicated significance of the difference between the group with only drug injected and the saline group.

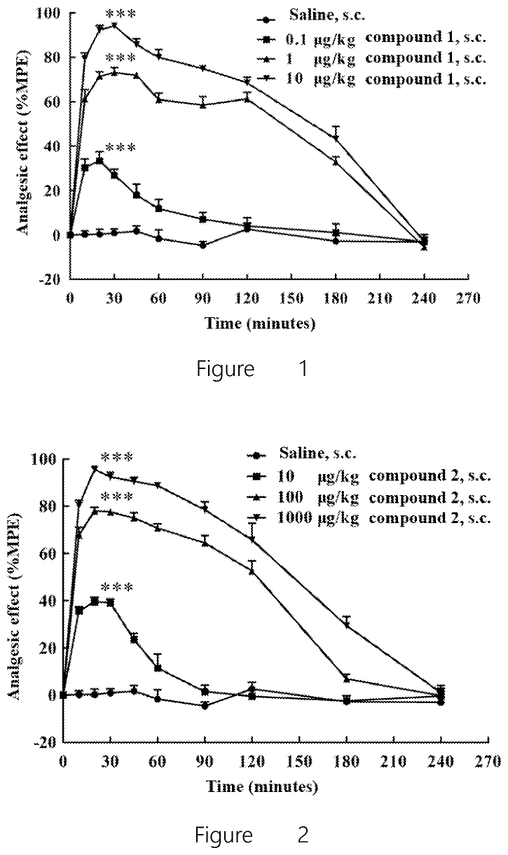

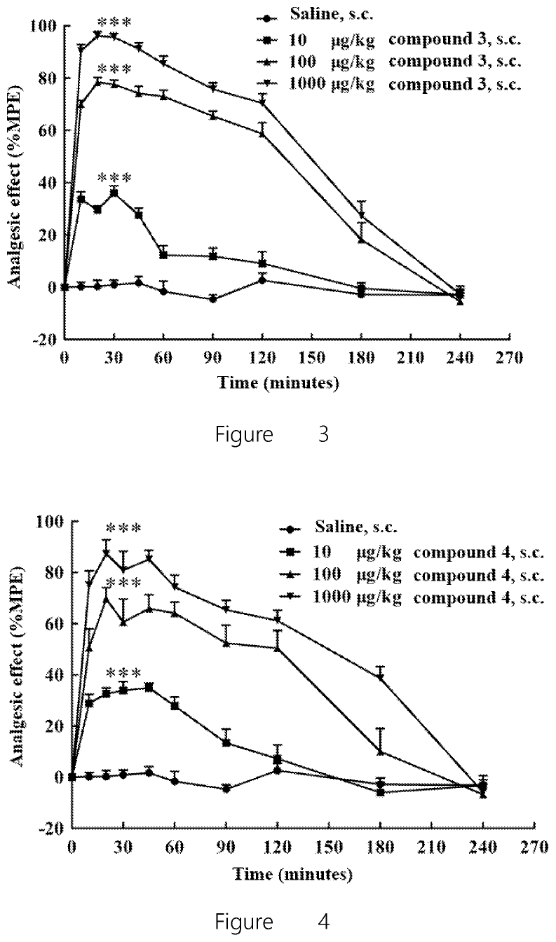

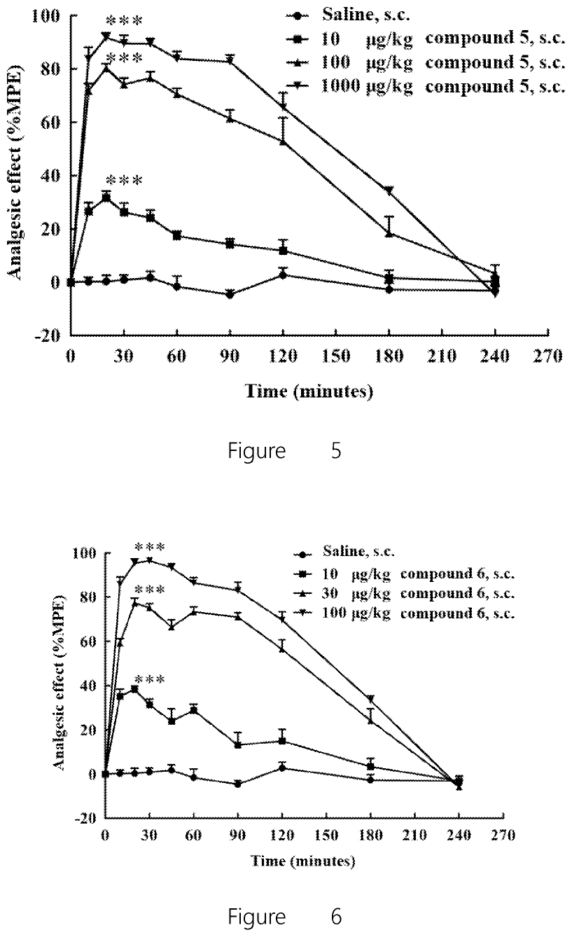

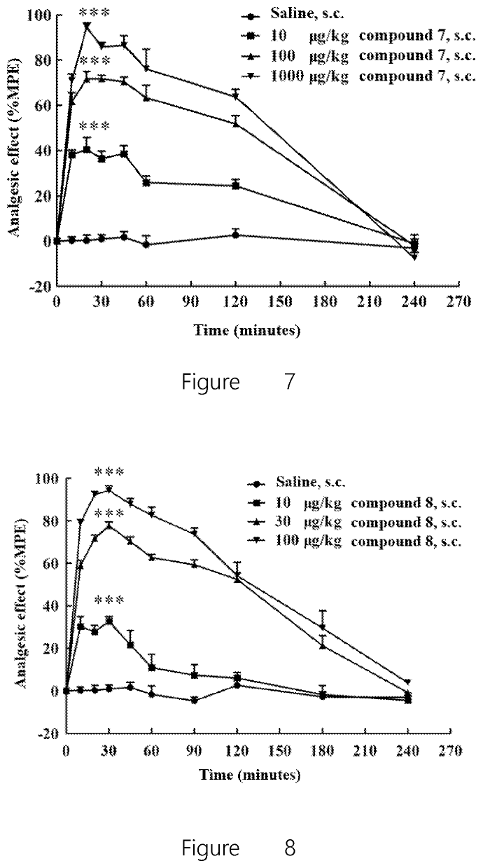

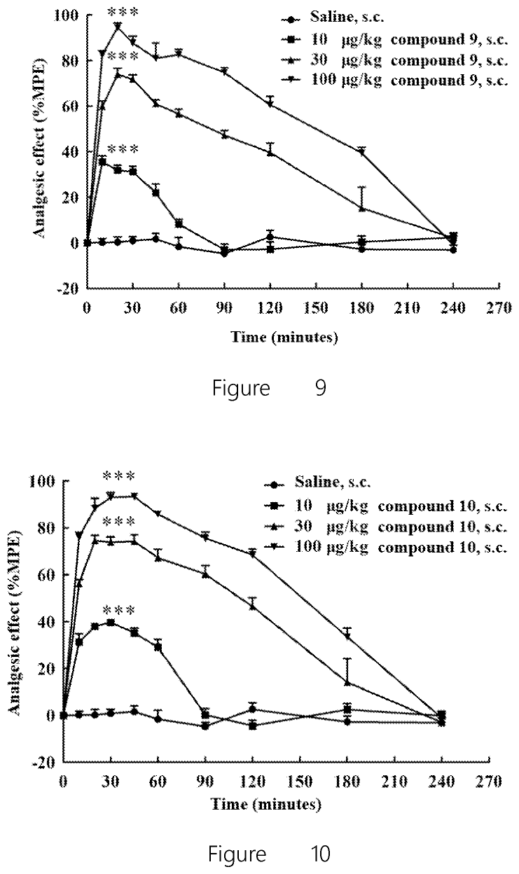

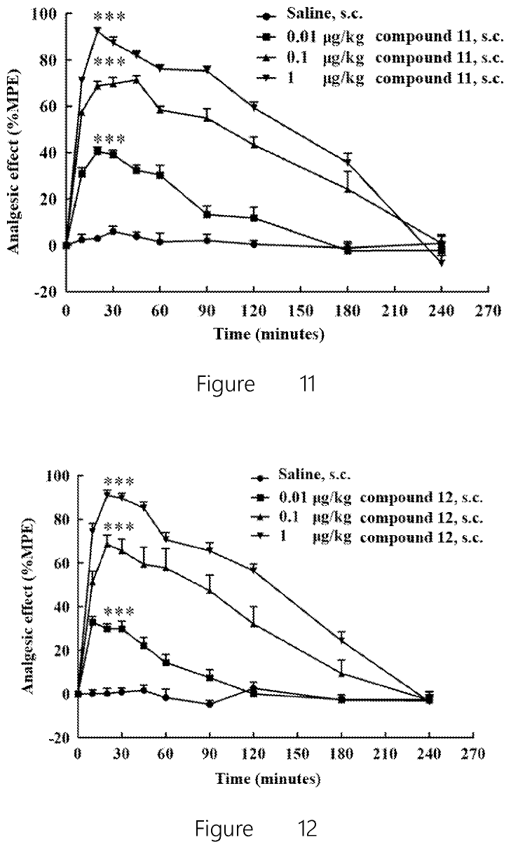

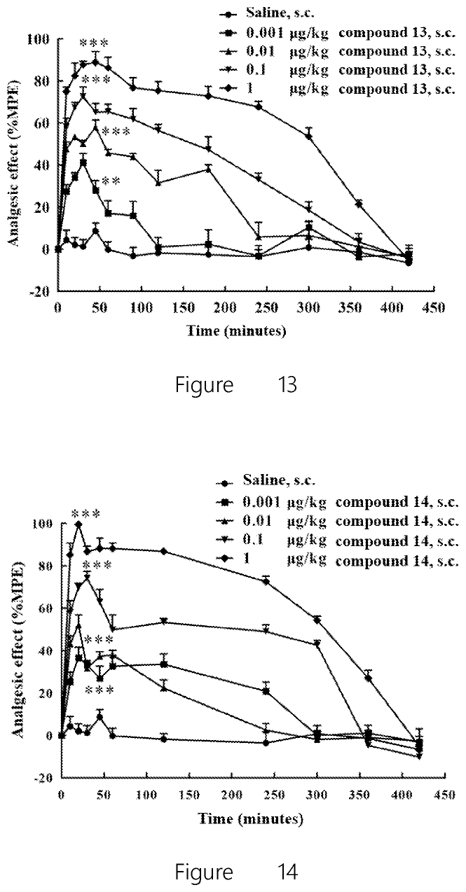

In the photothermal tail flick experiment of mice, the analgesic ED₅₀ of all cyclopeptide molecules and parent molecule DN-9 injected subcutaneously was shown in Table 5, and ED₅₀ value of the parent molecule DN-9 analgesic is 228 μg/kg. The ED₅₀ values of the 14 cyclopeptide molecules listed in Table 5 are significantly lower than the analgesic ED₅₀ of the parent peptide. Among them, the subcutaneous injection analgesic ED₅₀ of compounds 11-14 was at least 10000 times lower than that of the parent molecule DN-9. And the effective analgesic actuation duration is extended from 90 minutes of the parent molecule to 240 minutes. The subcutaneous analgesic dose-effect curve of the compounds are shown in FIGS. 1-14.

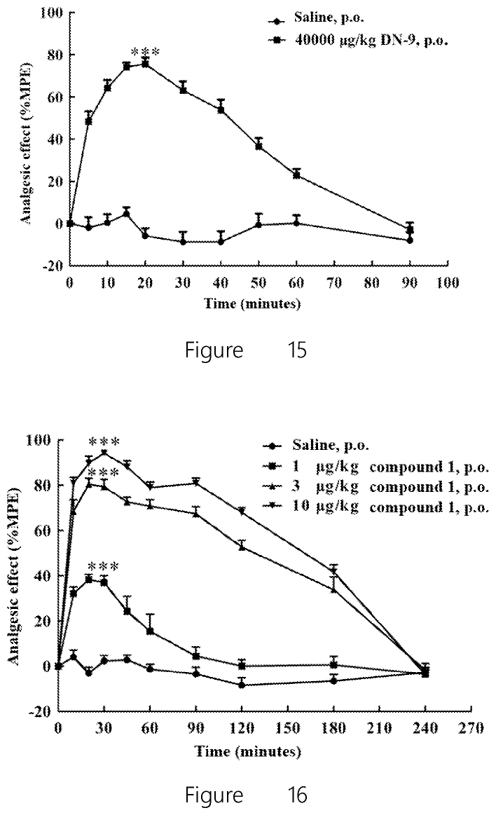

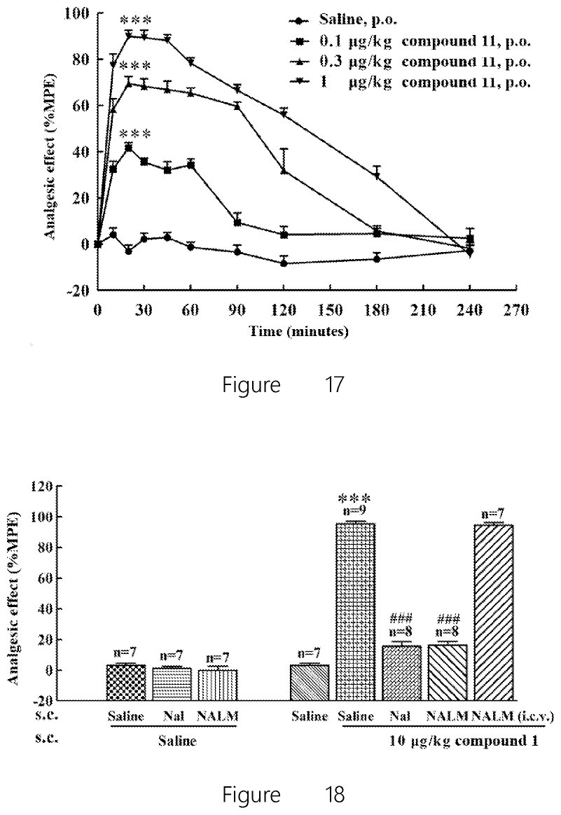

In the photothermal tail flick experiment of mice, the analgesic activity of oral administration of compounds 1-6 and 8-12 at the maximum doses was shown in Table 6. DN-9 only produces strong analgesic activity at a high-dose of 40 mg/kg. Compounds 1-6 and 8-12 have significantly better oral analgesic effect and analgesic activity than parent molecule (DN-9). Not only has the half analgesic dose decreased by hundreds of times, but the effective analgesic actuation duration has also been extended from 90 minutes of the parent molecule to 240 minutes. Especially the ED₅₀ values of compound 1 and compound 11 are 1.37 and 0.14 μg/kg, respectively, whose analgesic potency is far superior to that of the parent molecule. The oral analgesic diagrams of parent peptide DN-9, compounds 1 and 11 are shown in FIGS. 15-17.

TABLE 5
Analgesic activity generated by subcutaneous injection
of multi-target cyclopeptide molecules

| | ED₅₀value (μg/kg) |
|---|---|
| Compound | (95% confidence interval) |
| DN-9 | 228 (156.9 to 331.2) |
| Compound 1 | 0.32 (0.27 to 0.38) |
| Compound 2 | 17.9 (15.3 to 20.9) |
| Compound 3 | 25.4 (22.1 to 29.2) |
| Compound 4 | 30.3 (19.4 to 47.1) |
| Compound 5 | 23.2 (19.1 to 28.2) |
| Compound 6 | 15.8 (14.6 to 17.1) |
| Compound 7 | 19.6 (12.8 to 29.9) |
| Compound 8 | 15.0 (13.8 to 16.4) |
| Compound 9 | 16.0 (14.4 to 17.6) |
| Compound 10 | 15.0 (13.6 to 16.5) |
| Compound 11 | 0.02 (0.017 to 0.026) |
| Compound 12 | 0.03 (0.025 to 0.045) |
| Compound 13 | 0.0009 (0.0004 to 0.0016) |
| Compound 14 | 0.006 (0.003 to 0.01) |

TABLE 6
Analgesic activity generated by oral administration
of multi-target cyclopeptide molecules

| | maximum | Maximum | Maximum | ED₅₀value | |
|---|---|---|---|---|---|
| | analgesic | analgesic | analgesic | (μg/kg) | analgesic |
| | dose | time | MPE | (95% confidence | duration |
| Compound | (μg/kg) | (min) | (%) | interval) | (min) |
| DN-9 | 40000 | 20 | 75.3 ± 4.34 | — | 90 |
| Compound 1 | 10 | 30 | 94.2 ± 1.37 | 1.37 (1.22 to 1.54) | 240 |
| Compound 2 | 10000 | 30 | 91.9 ± 3.32 | — | 240 |
| Compound 3 | 10000 | 20 | 93.2 ± 1.47 | — | 240 |
| Compound 4 | 10000 | 20 | 82.5 ± 4.08 | — | 240 |
| Compound 5 | 10000 | 20 | 94.1 ± 2.17 | — | 240 |
| Compound 6 | 100 | 30 | 95.6 ± 3.19 | — | 240 |
| Compound 8 | 100 | 30 | 96.0 ± 3.12 | — | 240 |
| Compound 9 | 100 | 30 | 93.8 ± 1.67 | — | 240 |
| Compound 10 | 100 | 30 | 97.5 ± 1.20 | — | 240 |
| Compound 11 | 1 | 20 | 89.8 ± 2.63 | 0.14 (0.12 to 0.16) | 240 |
| Compound 12 | 10 | 20 | 95.1 ± 1.91 | — | 240 |

### Example 17: Blood-Brain Barrier Permeability Assay

The pharmacological evaluation experiment on the permeability of the blood-brain barrier detects whether a drug passes through the blood-brain barrier by injecting naloxone iodide. Naloxone iodide is a drug that cannot pass through the blood-brain barrier. In the experiment, naloxone iodide, naloxone, and compounds were injected at different sites, and the changes in the analgesic effect of the compounds were detected through photothermal tail flick experiment.

The experiment on the permeability mechanism of the blood-brain barrier was conducted using 21±2 g Kunming male mice, with an ambient temperature controlled at 22±2° C. The mice were allowed to eat and drink water freely. Both naloxone iodide (NALM) and naloxone (Nal) were administered 10 minutes in advance. Naloxone iodide was administered in three ways: intracerebroventricular (i.c.v.), subcutaneous (s.c.), and intraperitoneal (i.p.). Naloxone was administered in two ways: subcutaneous (s.c.) and intraperitoneal (i.p.). Different compounds were mainly administered at the subcutaneous (s.c.) and oral (p.o.) in peripheral levels, and then the analgesic changes after administration of antagonists and drugs were detected through photothermal tail flick experiments.

The experimental data was represented by MPE (maximum possible effect), MPE (%)=100×[(pain threshold after administration−basic pain threshold)/(10 seconds−basic pain threshold)]. The antagonistic effect of drugs was compared using the MPE value at the time point of the maximum analgesic effect of the relevant drug. The MPE data was represented by mean±S.E.M., and the difference in analgesic effects were analyzed using one-way ANOVA Bonferroni test. *P<0.05, **P<0.01, and ***P<0.001indicated significance of the difference between the group with only the relevant drug injected and the group with both antagonist and related compound injected.

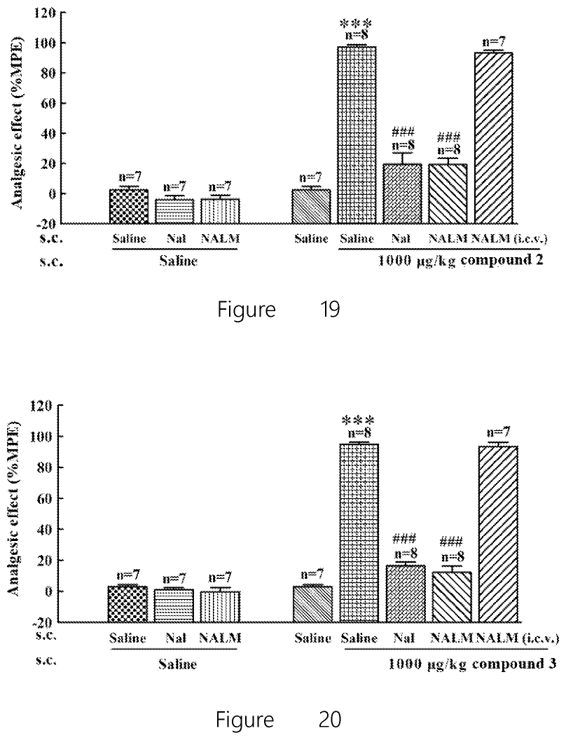

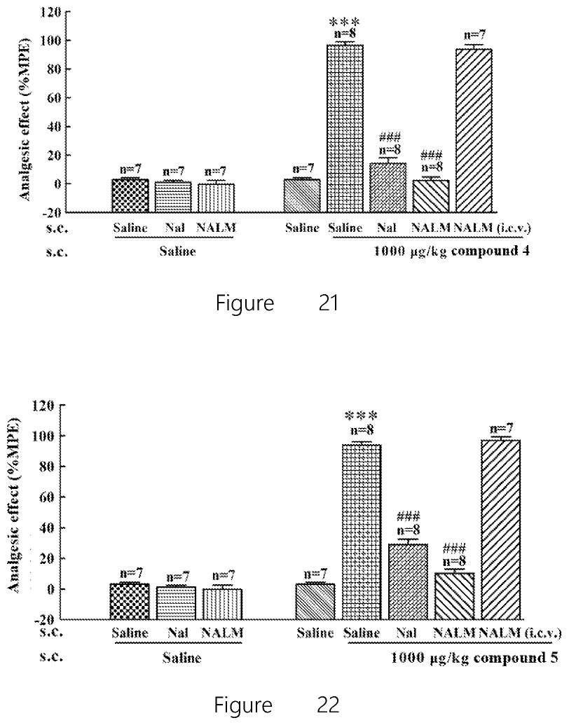

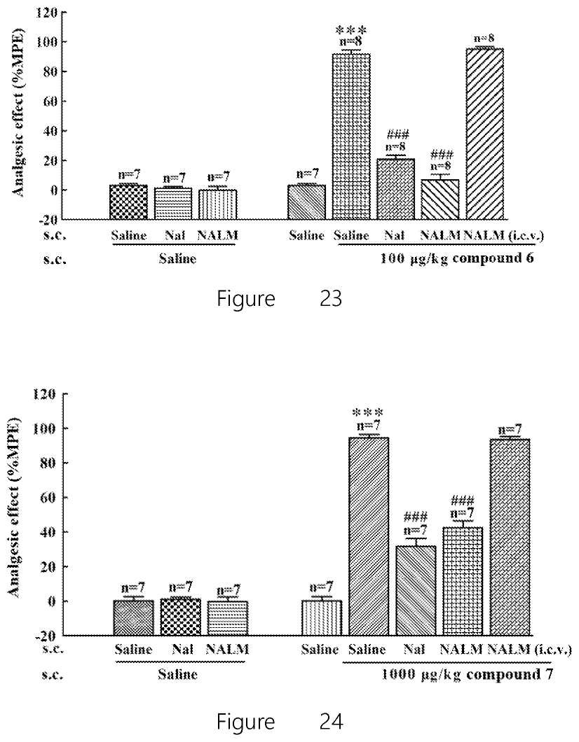

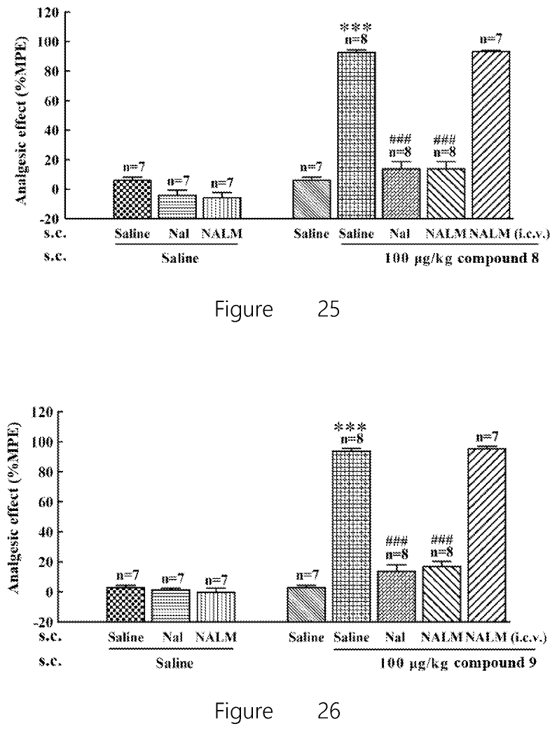

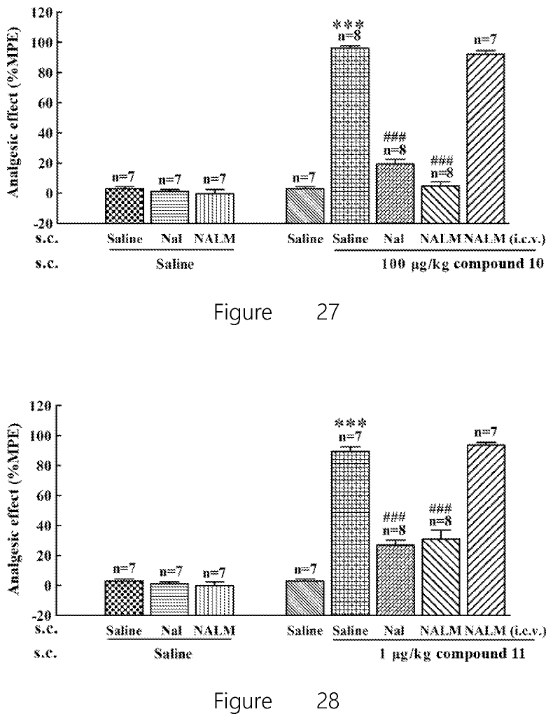

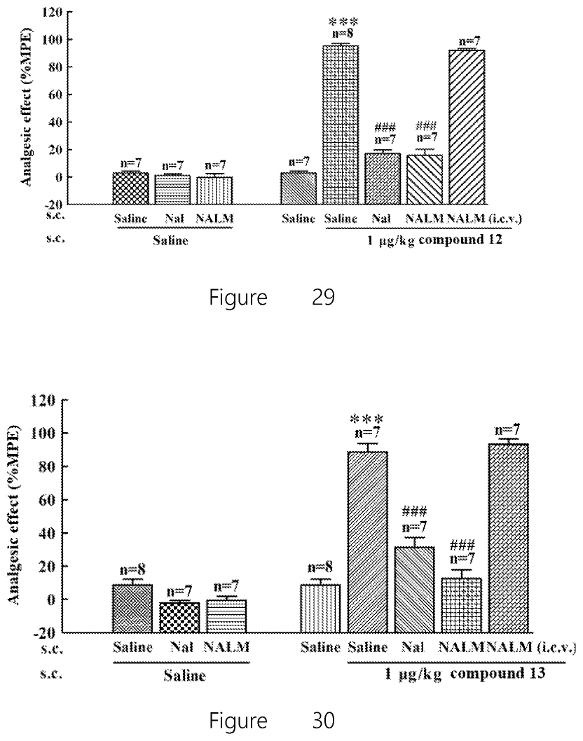

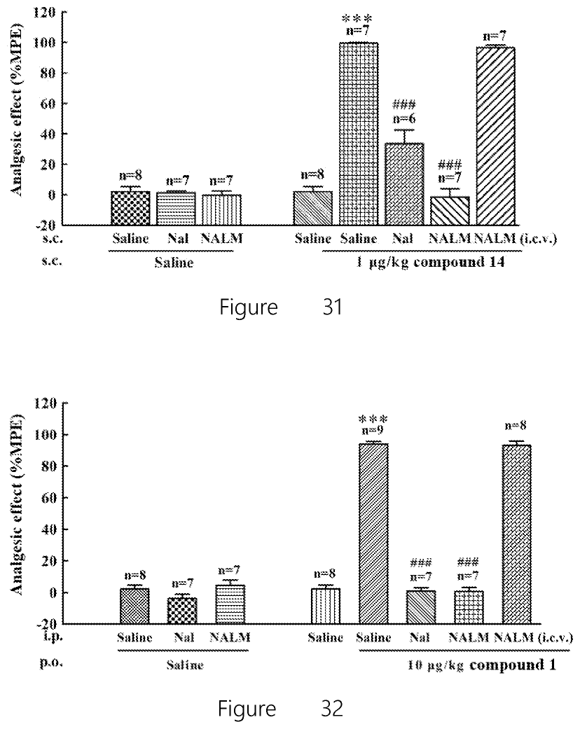

The results of the blood-brain barrier permeability were shown in Table 7. Intracerebroventricular injection of naloxone iodide cannot antagonize the analgesiat caused by subcutaneous injection of compounds 1-14. Subcutaneous injection of naloxone and naloxone iodide could antagonize the analgesiat caused by subcutaneous injection of compounds 1-14, indicating that neither compounds 1-14 could penetrate the blood-brain barrier. The specific results of the blood-brain barrier permeability study of compounds 1-14were shown in FIGS. 18-31.

TABLE 7
Blood-brain barrier permeability assays of the
cyclopeptide molecules of the present nvention

| | Blood-brain barrier |
|---|---|
| Compound | permeability |
| Compound 1 | Not permeable |
| Compound 2 | Not permeable |
| Compound 3 | Not permeable |
| Compound 4 | Not permeable |
| Compound 5 | Not permeable |
| Compound 6 | Not permeable |
| Compound 7 | Not permeable |
| Compound 8 | Not permeable |
| Compound 9 | Not permeable |
| Compound 10 | Not permeable |
| Compound 11 | Not permeable |
| Compound 12 | Not permeable |
| Compound 13 | Not permeable |
| Compound 14 | Not permeable |

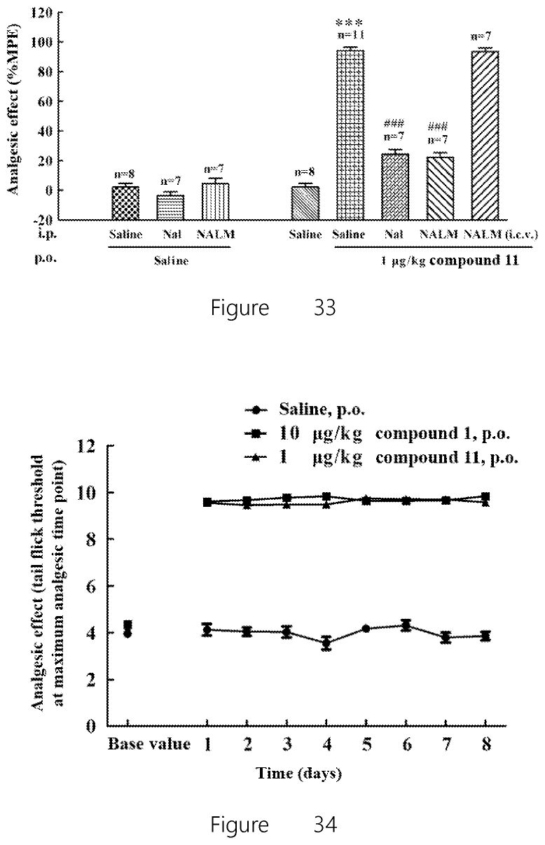

The results of blood-brain barrier permeability after oral administration of compounds 1 and 11 were shown in FIGS. 32 and 33. Intracerebroventricular injection of naloxone iodide cannot antagonize the analgesiat caused by oral injection of compounds 1 and 11, while intraperitoneal injection of naloxonean and naloxone antagonize the analgesiat caused by oral injection of compounds 1 and 11, indicating that compounds 1 and 11 cannot penetrate the blood-brain barrier. This suggests that the compounds of the present invention have low side effects on the central nervous system.

### Example 18. Analgesic Tolerance Assay for Compounds 1 and 11

The analgesic tolerance experiment was conducted by orally injecting compounds 1 and 11 for 8 consecutive days, and then identifying the changes in the analgesic tail flick threshold from the first day to the eighth day through a photothermal tail flick experiment, thus evaluating the pharmacological activity of compounds 1 and 11 in the present invention in terms of analgesic tolerance.

21±2 g Kunming male mice were selected for the experiment, and the mice were eat freely. Generally, the basic pain threshold of mice was measured on the first day, followed by oral injection of compounds 1 and 11 for 8 consecutive days. The pain threshold at different time points was measured on the first day, and only the pain threshold at the highest analgesic point was measured on the following 7 days. The injection of traditional opioid drugs into mice generally resulted in the decrease in the analgesic threshold on the 3rd or 4th day, indicating the onset of analgesic tolerance.

The experimental data was represented by tail flick time. The analgesic tolerance of drugs was compared using the tail flick latency at the maximum analgesic effect time point of different compounds. The tail flick latency data was represented by mean±S.E.M. The differences of analgesic effects of subcutaneous administration for eight days in mice were analyzed using one-way ANOVA (Tukey HSD test). *P<0.05, **P<0.01, and ***P<0.001 indicated a significant difference in analgesic effects compared to the injection of the drug on the first day. As shown in FIG. 34, after 8 consecutive days of oral injection, there was no change in the saline group, and there was no analgesic tolerance observed after 8 consecutive days of oral injection of compounds 1 and 11.

### Example 19. Constipation Side Effect Assay of Oral Injection of Compounds 1 and 11

Constipation is a common side effect of opioid drugs. Therefore, the impact of drugs on gastrointestinal motility is generally evaluated through the side effects of constipation.

26±2 g Kunming male mice were selected. First, the mice were starved and placed in a box without padding. The mice were not allowed to eat, but could drink water freely. The mice were starved for 16 hours then weighted, and administered orally (p.o.). After 15 minutes of administration, pre-prepared activated carbon suspension (a physiological saline suspension containing 5% activated carbon and 10% arabic gum) was orally infused into the stomach at a volume of 0.1 mL/10 g. After 30 minutes of gavage, the mice were sacrificed through cervical dislocation. The mice were dissected immediately and the entire length of the small intestine was taken from the pylorus of the stomach to the cecum. Then the total length of the small intestine and the length of carbon powder movement were measured.

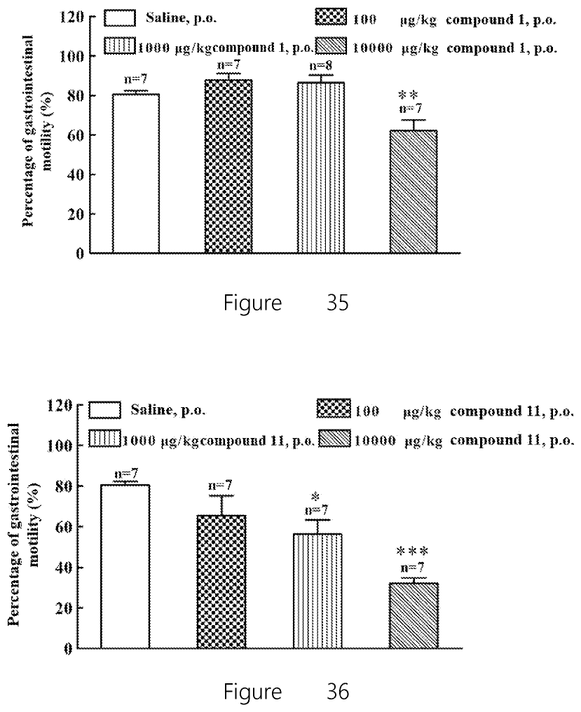

The experimental data of gastrointestinal motility was represented by a percentage of gastrointestinal motility, and the specific calculation method was represented by a percentage of the distance the carbon powder moved divided by the total length of the small intestine. The data was represented by the mean±S.E.M. of the percentage of gastrointestinal motility, and the differences between compounds and saline were analyzed using one-way ANOVA (Dunnett test). *P<0.05, **P<0.01, and ***P<0.001indicated significance of the difference between only saline injected and related compounds injected. The results of gastrointestinal motility experiments for compounds 1 and 11 were shown in FIGS. 35-36.

In FIG. 35, physiological saline, 100, 1000 and 10000 μg/kg of compound 1 were orally injected, respectively. The percentage of gastrointestinal inhibition was 84.54% in the saline group, and 87.82%, 86.46%, and 62.25% in the drug administration groups, respectively. The ED₅₀ of gastrointestinal inhibition is 37050 μg/kg. Oral analgesic ED₅₀ of compound 1 is 1.37 μg/kg, that is, ED₅₀ of the gastrointestinal inhibition of side effect of constipation is 27044 times ED₅₀ of analgesic. There is no side effect of constipation within the effective dose range of analgesia.

In FIG. 36, physiological saline, 100, 1000 and 10000 μg/kg of compound 11 were orally injected, respectively. The percentage of gastrointestinal inhibition was 80.54% in the saline group, and 65.42%, 56.35%, and 32.12% in the drug administration groups, respectively. The ED₅₀ of gastrointestinal inhibition was 1239 μg/kg. Oral analgesic ED₅₀ of compound 11 is 0.14 μg/kg, that is, ED₅₀ of the gastrointestinal inhibition of side effect of constipation is 8850 times ED₅₀ of analgesic. There is no side effect of constipation within the effective dose range of analgesia.

### Example 20. Addiction Side Effect Assay of Oral Injection of Compounds 1 and 11

The addiction evaluation of compounds 1 and 11 was conducted through open field test, conditioned place preference (CPP), and naloxone withdrawal experiments. Injecting opioid drugs promotes the release of dopamine and enhances the locomotor activity of mice. Therefore, locomotor activity is often associated with the evaluation of addiction in mice.

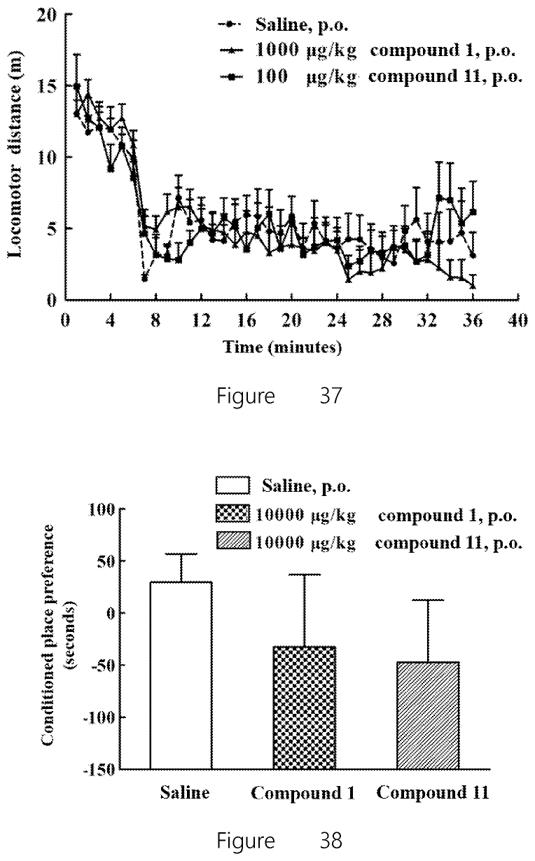

Open field test was consisted of an uncapped 50×50×40 cm black organic glass box and a set of motion monitoring system. 21±2 g Kunming male mice were selected for the experiment. The room temperature was controlled between 22±1° C., otherwise, high or low temperatures would affect the movement of the mice. Before the experiment began, the box was wipped with alcohol to remove the odor inside and avoid the odor from affecting the locomotor activity of the next mouse. Before the experiment, the basic locomotor activity of the mouse in 30 minutes was recorded, followed by oral (p.o.) injection of physiological saline, 1000 μg/kg of compound 1, and 100 μg/kg of compound 11. The locomotor activity of mice within 150 minutes was recorded. The locomotor activity of mouse was represented by the total distance traveled, which is the total distance±S.E.M. The difference between the compound group and saline control group was statistically analyzed using one-way ANOVA (Bonferroni test). *P<0.05, **P<0.01, and ***P<0.001 indicated significance of difference between saline and compound. The results were shown in FIG. 37.

Conditioned place preference experiment (CPP) was conducted in a device consisting of three organic glass boxes, wherein two large boxes (20×20×20 cm) on either side separated by a small box (5×20×20 cm) in the middle. A small door of 5×5 was at the bottom of two large boxes for mice to enter and exit, and the doors can be closed. A box on one side was white, with a rough wire mesh as the bottom and a light intensity of 50 lux. A box on other side was black, with a smooth bottom and a light intensity of 20 lux. 25±5 g male mice were selected for the experiment, the room temperature was 22±1° C. On the first day of the experiment, the mice were first screened, allowing them to shuttle freely between two boxes for 15 minutes. The time the mice stayed in one box was recorded, and mice that stayed for more than 9 minutes were removed. Mice without preference/aversion were selected. For the next 3 days, physiological saline or compound 1 (10000 μg/kg) and compound 11 (10000 μg/kg) were administrated orally (p.o.) continuously. The small door in the middle was closed, and both the drug administration group and the physiological saline group were divided into two groups. One group of mice was given saline in a white box in the morning and given drug in a black box in the afternoon. The other group of mice was given saline in a black box in the morning, and given drug in a white box in the afternoon. The mice adapted in the box for 45 minutes and continued training for 3 days. On the 5th day, CPP performance was measured. As on the first day, the time stay in each box was measured for the mice after administration with the duration of 15 minutes. The inter group differences between different drug treatments in the CPP experiment and the differences in the number of jumps in the naloxone withdrawal experiment was statistically analyzed using paired t-tests. *P<0.05, **P<0.01, and ***P<0.001 indicated significance of differences between the drug treatment group and the saline group. The results were shown in FIG. 38.

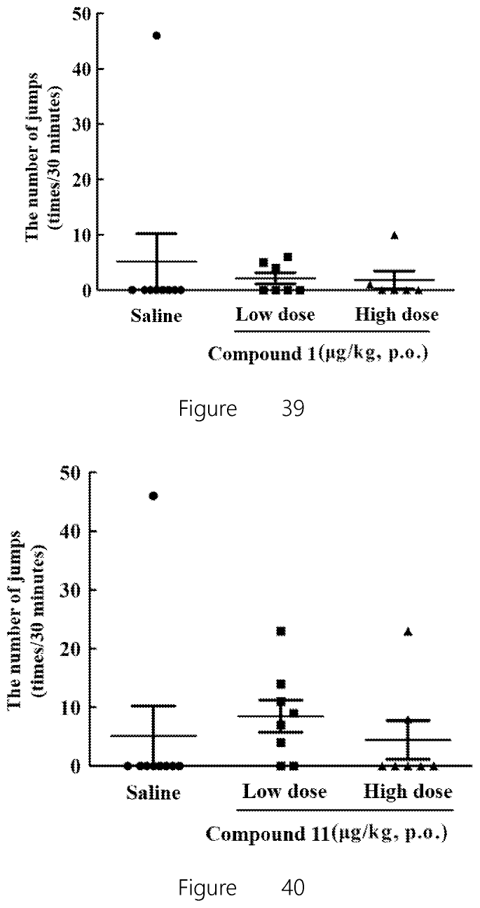

The naloxone withdrawal test is a classic experiment for evaluating drug physiological addiction. The experimental method is based on Venetia Zachariou (2003). The specific experimental method was compounds 1 and 10 were orally administered (p.o.) every 8 hours with the dosage of the experimental drug gradually increased. As reference for morphine (20, 40, 60, 80, 100, 100, 100 mg/kg), the analgesic ED₅₀ of morphine is 1.68 mg/kg, that is, the dosage of morphine is 10, 20, 30, 40, 50, 50, and 50 times its analgesic ED₅₀. Therefore, referring to the experimental method, the oral ED₅₀ of compound 1 is 1.37 μg/kg, therefore the low doses of oral administration were approximately 20, 40, 60, 80, 100, 100, and 100 μg/kg, respectively; in addition, the high-dose group was administered at doses of 100, 200, 300, 400, 500, 500, and 500 times analgesic ED₅₀ of compound 1, with doses of approximately 200, 400, 600, 800, 1000, 1000, and 1000 μg/kg, respectively. The analgesic ED₅₀ of compound 11 is 0.14 μg/kg, therefore the oral dosage was approximately 2, 4, 6, 8, 10, 10, and 10 μg/kg, respectively. High doses of compound 11 were also administered with doses of 20, 40, 60, 80, 100, 100, and 100 μg/kg, respectively. Two hours after the last administration, mice were intraperitoneally injected with 10 mg/kg of naloxone. Then the mouse was immediately placed in an opaque barrel-shaped structure with an inner diameter of 9 cm and a height of 32 cm, and the number of jumps in mice within 30 minutes was recorded. The experimental results were represented by the number of jumps in mice, which is the number of jumps±S.E.M. The difference between the compound group and saline control group was statistically analyzed using one-way ANOVA (Bonferroni test). *P<0.05, **P<0.01, and ***P<0.001 indicated significance of differences between saline and compound. The results were shown in FIGS. 39-40.

As shown in FIG. 37, in the open field test, physiological saline, 1000 μg/kg of compound 1, and 100 μg/kg of compound 11 were orally injected. The locomotor distance of the saline group was 131.1±33.93 m. The locomotor distance of compound 1 was 109.4±32.16 m. The locomotor distance of compound 11 was 127.9±23.49 m. Compared with the saline control group, there is no change in the locomotor activity of the mice.

As shown in FIG. 38, in the mouse conditioned place preference experiment (CPP), physiological saline, 10000 μg/kg of compound 1, and 10000 μg/kg of compound 11 were administered orally. There is no phenomenon of conditioned place preference.

As shown in FIGS. 39 and 40, in the naloxone withdrawal experiment, physiological saline, compound 1 and compound 11 were orally injected at low and high doses. The number of jumps in the saline group is 5. The number ofjumps for both low-dose and high-dose compound 1 is 1. The number of jumps for low-dose and high-dose compound 11 is 9 and 4, respectively. Compared with the saline control group, the mice did not show withdrawal symptoms.

All documents mentioned herein are incorporated by reference in this application as if each document were individually incorporated by reference. In addition, it should be understood that after reading the above teaching content of the present invention, those skilled in the art can make various changes or modifications to the present invention, and these equivalent forms also fall within the scope defined by the appended claims of the present application.

1-14. (canceled)

15. A multi-target cyclopeptide molecule for opioid receptors and neuropeptide FF receptors, or a pharmaceutically acceptable salt thereof, wherein the structure of the cyclopeptide molecule is shown in formula I:
Tyr-c^{[2,5]}[Xaa2-Gly-NMe-Phe-Xaa5]-Xaa6-Xaa7-Arg-Xaa9-NH₂ (I)
wherein,
Xaa2 is Lys, D-Lys, D-Asp, D-Glu, D-Orn, D-Dab, or D-Dap;
Xaa5 is Asp, D-Asp, Glu, or Lys;
Xaa6 is Pro or Gly;
Xaa7 is Gln β-Ala or Aib;
Xaa9 is Phe or Cha;
c^{[2,5]} represents the presence of a cycle-forming covalent bond between the two amino acid residues Xaa2 and Xaa5 in the amino acid sequence.

16. The multi-target cyclopeptide molecule for opioid receptors and neuropeptide FF receptors, or a pharmaceutically acceptable salt thereof according to claim 15, wherein the cycle-forming covalent bond between the two amino acid residues Xaa2 and Xaa5 includes the formation of amide bond through dehydration condensation.

17. The multi-target cyclopeptide molecule for opioid receptors and neuropeptide FF receptors, or a pharmaceutically acceptable salt thereof according to claim 15, wherein the structure of the cyclopeptide molecule is shown in formula II:
```
(II)
Tyr-Xaa2~L0~Xaa5-Xaa6-Xaa7-Arg-Xaa9-NH₂
        | |
        Gly-NMePhe
```
wherein,
Xaa2, Xaa5, Xaa6, Xaa7, and Xaa9 as defined in claim 15;
‘˜L0˜’ represents the cycle-forming covalent bond between the two amino acid residues Xaa2 and Xaa5.

18. The multi-target cyclopeptide molecule for opioid receptors and neuropeptide FF receptors, or a pharmaceutically acceptable salt thereof according to claim 15, wherein the cyclopeptide molecule is selected from the following compound:
compound 1:
Tyr-c^{[2,5]}[D-Lys-Gly-NMe-Phe-Asp]-
Pro-Gln-Arg-Phe-NH₂,
compound 2:
Tyr-c^{[2,5]}[D-Lys-Gly-NMe-Phe-Glu]-
Pro-Gln-Arg-Phe-NH₂,
compound 3:
Tyr-c^{[2,5]}[Lys-Gly-NMe-Phe-Asp]-
Pro-Gln-Arg-Phe-NH₂,
compound 4:
Tyr-c^{[2,5]}
[Lys-Gly-NMe-Phe-D-Asp]-Pro-Gln-
Arg-Phe-NH₂,
compound 5:
Tyr-c^{[2,5]}
[D-Lys-Gly-NMe-Phe-D-Asp]-Pro-Gln-
Arg-Phe-NH₂,
compound 6:
Tyr-c^{[2,5]}
[D-Asp-Gly-NMe-Phe-Lys]-Pro-Gln-Arg-
Phe-NH₂,
compound 7:
Tyr-c^{[2,5]}[D-Glu-Gly-NMe-Phe-Lys]-Pro-
Gln-Arg-Phe-NH₂,
compound 8:
Tyr-c^{[2,5]}[D-Orn-Gly-NMe-Phe-Asp]-Pro-
Gln-Arg-Phe-NH₂,
compound 9:
Tyr-c^{[2,5]}[D-Dab-Gly-NMe-Phe-Asp]-Pro-
Gln-Arg-Phe-NH₂,
compound 10:
Tyr-c^{[2,5]}[D-Dap-Gly-NMe-Phe-Asp]-Pro-
Gln-Arg-Phe-NH₂,
compound 11:
Tyr-c^{[2,5]}[D-Orn-Gly-NMe-Phe-Glu]-Pro-
Gln-Arg-Phe-NH₂,
compound 12:
Tyr-c^{[2,5]}
[D-Lys-Gly-NMe-Phe-Asp]-Gly-Gln-Arg-
Phe-NH₂,
compound 13:
Tyr-c^{[2,5]}[D-Lys-Gly-NMe-Phe-Asp]-Pro-
β-Ala-Arg-Cha-NH₂;
and
compound 14:
Tyr-c^{[2,5]}[D-Lys-Gly-NMe-Phe-Asp]-Pro-
Aib-Arg-Cha-NH₂.

19. The multi-target cyclopeptide molecule for opioid receptors and neuropeptide FF receptors, or a pharmaceutically acceptable salt thereof according to claim 15, wherein the cyclopeptide molecule as shown in formula I has one or more features selected from the group consisting of:
(1) the cyclopeptide molecule as shown in formula I is a dual target agonist for opioid receptors and NPFF receptors;
(2) the analgesic activity of subcutaneous injection of cyclopeptide molecule as shown in formula I is more than 10 times that of DN-9;
(3) the oral analgesic activity of the cyclopeptide molecule as shown in formula I is more than 3 times that of DN-9;
(4) the cyclopeptide molecule as shown in formula I does not pass through the blood-brain barrier;
(5) the cyclopeptide molecule as shown in formula I has not shown analgesic tolerance after continuous oral administration for more than 5 days;
(6) the cyclopeptide molecule as shown in formula I does not have constipation side effect;
(7) the cyclopeptide molecule as shown in formula I does not have addictive side effect.

20. The multi-target cyclopeptide molecule for opioid receptors and neuropeptide FF receptors, or a pharmaceutically acceptable salt thereof according to claim 19, wherein the cyclopeptide molecule as shown in formula I has one or more features selected from the group consisting of:
(1) the analgesic activity of subcutaneous injection of cyclopeptide molecule as shown in formula I is more than 100 times that of DN-9;
(2) the oral analgesic activity of the cyclopeptide molecule as shown in formula I is more than 10 times that of DN-9;
(3) the cyclopeptide molecule as shown in formula I has not shown analgesic tolerance after continuous oral administration for more than 8 days.

21. The multi-target cyclopeptide molecule for opioid receptors and neuropeptide FF receptors, or a pharmaceutically acceptable salt thereof according to claim 19, wherein the cyclopeptide molecule as shown in formula I has one or more features selected from the group consisting of:
(1) the analgesic activity of subcutaneous injection of cyclopeptide molecule as shown in formula I is more than 1000 times that of DN-9;
(2) the oral analgesic activity of the cyclopeptide molecule as shown in formula I is more than 500 times that of DN-9.

22. A prepartion method for the multi-target cyclopeptide molecule for opioid receptors and neuropeptide FF receptors, or a pharmaceutically acceptable salt thereof according to claim 15, comprising the steps of:
(a) using liquid phase synthesis method and/or solid phase synthesis method, synthesising the peptide chain according to the amino acid sequence corresponding to the structural formula I, thereby obtaining linear peptide chain; and
(b) cyclizing the linear peptide chain to obtain the multi-target cyclopeptide molecule for opioid receptors and a neuropeptide FF (NPFF) receptor, or a pharmaceutically acceptable salt thereof.

23. The prepartion method for the multi-target cyclopeptide molecule for opioid receptors and neuropeptide FF receptors, or a pharmaceutically acceptable salt thereof according to claim 22, wherein in step (a), peptide chain synthesis is carried out from the C-terminus of the amino acid sequence to the N-terminus or from the N-terminus of the amino acid sequence to the C-terminus.

24. The prepartion method for the multi-target cyclopeptide molecule for opioid receptors and neuropeptide FF receptors, or a pharmaceutically acceptable salt thereof according to claim 22, wherein step (b) includes that the side chains of Xaa2 and Xaa5 are cyclized to form a cycle-forming covalent bond between the two amino acid residues Xaa2 and Xaa5.

25. The prepartion method for the multi-target cyclopeptide molecule for opioid receptors and neuropeptide FF receptors, or a pharmaceutically acceptable salt thereof according to claim 22, wherein the solid phase synthesis method comprises the process steps selected from the group consisting of: pretreatment of the solid phase carrier, amino acid condensation, extension of the peptide chain, compression, drying, and cutting of the peptide, extraction, purification, and the combinations thereof.

26. The prepartion method for the multi-target cyclopeptide molecule for opioid receptors and neuropeptide FF receptors, or a pharmaceutically acceptable salt thereof according to claim 25, wherein the solid phase carrier is amino resin.

27. The prepartion method for the multi-target cyclopeptide molecule for opioid receptors and neuropeptide FF receptors, or a pharmaceutically acceptable salt thereof according to claim 25, wherein condensation reagents used in the amino acid condensation step include a combination of HOBt, HBTU, and DIEA.

28. The prepartion method for the multi-target cyclopeptide molecule for opioid receptors and neuropeptide FF receptors, or a pharmaceutically acceptable salt thereof according to claim 22, wherein cyclization reagent used in cyclization step includes a combination of PyBOP and DIEA.

29. A pharmaceutical composition, comprising:
(a) multi-target cyclopeptide molecule for opioid receptors and neuropeptide FF receptors or a pharmaceutically acceptable salt thereof according to claim 15 as active ingredient; and
(b) pharmaceutically acceptable carriers and/or excipients.

30. The pharmaceutical composition according to claim 29, wherein the formulation of the pharmaceutical composition is an injection.

31. The pharmaceutical composition according to claim 29, wherein the pharmaceutical composition is administered by means selected from the group consisting of oral administration, percutaneous administration, intrathecal administration, intravenous administration, intramuscular administration, local administration, nasal administration, etc.

32. A method for analgesia, comprising the steps of administering a safe and effective amount of multi-target cyclopeptide molecule for opioid receptors and neuropeptide FF receptors or a pharmaceutically acceptable salt thereof according to claim 15.
